SEQUENCE LISTING (5 sequences)

SEQ ID NO: 1 - DNA, 57 bp, Artificial sequence
  beta-catenin-antisense promoter-linked primer
ccagtgaattgtaatacgactcactataggcgctctgaagacagtctgtcgtgatgg

SEQ ID NO: 2 - DNA, 24 bp, Artificial sequence
  beta-catenin-sense primer
atggcaacccaagctgacttgatc

SEQ ID NO: 3 - DNA, 24 bp, Artificial sequence
  A sense primer for construction of a RNA/DNA hybrid targeting HIV
ggatgncngncnccttgttggtcc

SEQ ID NO: 4 - DNA, 80 bp, Artificial sequence
  Partial Mus musculus tyrosinase (tyr) sense DNA (sDNA) sequence
gtgctcaggcaacttcatgggtttcaactgcggaaactgtaagtttggatttgggggcccaaattgtacagagaagcgag

SEQ ID NO: 5 - RNA, 80 nt, Artificial sequence
  complementary antisense RNA (aRNA) to SEQ ID.4
cucgcuucucuguacaauuugggcccccaaauccaaacuuacaguuuccgcaguugaaacccaugaaguugccugagcac

(19) United States
(12) Patent Application Publication
(10) Pub. No.: US 2004/0087526 A1
(43) Pub. Date: May 6, 2004

(54) Therapeutically useful compositions of DNA-RNA hybrid duplex constructs

(76) Inventors: Shi-Lung Lin, Alhambra, CA (US); Henry H. Ji, San Diego, CA (US)

Correspondence Address: Raymond Y. Chan, Suite 128, 108 N. Ynez Ave., Monterey Park, CA, 91754, US

(21) Appl. No.: 10/393,450

(22) Filed: Mar. 19, 2003

Related U.S. Application Data
(63) Continuation-in-part of application No. 10/052,486, filed on Jan. 18, 2002.
(60) Provisional application No. 60/351,183, filed on Nov. 12, 2001.

(51) Int. Cl. A61K 48/00; C12Q 1/68; C12P 19/34

(52) U.S. Cl. 514/44; 435/6; 435/91.2

(57) ABSTRACT

The present invention provides novel compositions and methods for suppressing the expression of a targeted gene using RNA-DNA hybrid constructs. The invention further provides novel methods and compositions for generating or producing RNA-DNA hybrids, whose quantity is high enough to be used for the invention's gene silencing transfection and possibly in therapeutics applications. This improved RNA-polymerase chain reaction method utilizes thermocycling steps of promoter-linked DNA or RNA template synthesis, in vitro transcription and then reverse transcription to bring up the amount of RNA-DNA hybrids up to two thousand folds within one round of the above procedure for specific gene silencing.

### CROSS REFERENCE OF RELATED APPLICATION

This is a Continuation-In-Part application claiming priority to a non-provisional application, application Ser. No. 10/052,486, filed on Jan. 22, 2002, entitled GENE SILENCING USING SENSE DNA AND ANTISENSE RNA HYBRID CONSTRUCTS, and a provisional application, application No. 60/351,183, filed on Nov. 12, 2001, entitled GENE SILENCING USING SENSE DNA AND ANTISENSE RNA HYBRID CONSTRUCTS, which is hereby incorporated by reference as if fully set forth herein.

### BACKGROUND OF THE PRESENT INVENTION

1. Field of Invention

The present invention generally relates to the field of compositions and methods used in altering the characteristics of eukaryotic cells. In particular, it relates to suppression or inhibition of specific gene functions by means of specific intracellular RNA species degradation or decay, gene transcript knockout, posttranscriptional gene silencing (PTGS), and/or RNA interference (RNAi) in eukaryotic cells.

2. Description of Related Arts

Gene silencing, specific RNA molecule degradation or breakdown such as specific degradation or breakdown of gene mRNA transcripts, tRNAs, hnRNAs, viral and other pathogen RNA genomes and other RNAs, or inhibiting the expression of a gene holds great therapeutic and diagnostic promise. An example of this approach is the use of antisense technology to inhibit gene expression in vitro and in vivo. Antisense technology involves the introduction into cell of an oligonucleotide sequence that is complementary to a target messenger RNA sequence in the cell. Many problems remain, however, with development of effective antisense technology. For example, single-stranded DNA antisense oligonucleotides exhibit only short-term effectiveness and are usually toxic at the doses required for biological effectiveness. Similarly, the use of single-stranded antisense RNAs has also proved ineffective due to its fast degradation and structural instability.

Other approaches to inhibiting or quelling specific gene activities are by means of posttranscriptional gene silencing (PTGS) and RNA interference (RNAi) phenomena, which have been applied to a variety of in-vivo systems, including plants, Drosophila melanogaster, Caenorhabditis elegans, and mouse. (Grant, S. R. (1999) Cell 96, 303-306), (Kennerdell, J. R. and Carthew, R. M. (1998) Cell 95, 1017-1026, Misquitta, L. and Paterson, B. M. (1999) Proc. Natl. Acad. Sci. USA 96, 1451-1456, and Pal-Bhadra, M., Bhadra, U., and Birchler, J. A. (1999) Cell 99, 35-46), (Tabara, H., Sarkissian, M., Kelly, W. G., Fleenor, J., Grishok, A., and Timmons, L. (1999) Cell 99, 123-132, Ketting, R. F., Haverkamp, T. H., van Luenen, H. G., and Plasterk, R. H. (1999) Cell 99, 133-141, Fire, A., Xu, S., Montgomery, M. K., Kostas, S. A., Driver, S. E., and Mello, C. C. (1998) Nature 391, 806-811 and Grishok, A., Tabara, H., and Mello, C. C. (2000) Science 287, 2494-2497), zebrafish (Wargelius, A., Ellingsen, S., and Fjose, A. (1999) Biochem. Biophys. Res. Commun. 263, 156-161) (Wianny, F. and Zernicka-Goetz, M. (2000) Nature Cell Biol. 2, 70-75). (These publications and all other cited publications and patents in this application are hereby incorporated by reference as if fully set forth herein.)

In general, the PTGS phenomena involves the transfection of a plasmid-like DNA structure or double stranded DNA (transgene) into cells, while the RNAi phenomena involves the transfection of double-stranded RNA (dsRNA) into the cells. These phenomena appear to evoke an intracellular sequence-specific RNA degradation process, affecting all highly homologous transcripts, called cosuppression. It has been proposed that such cosuppression results from the generation of small interfering RNA (siRNA) products (21˜25 nucleotide bases) by an RNA-directed RNA polymerase (RdRp) and/or a ribonuclease (RNase) activity on an aberrant RNA template, derived from the transfection of nucleic acids or viral infection. (Grant, supra, Ketting et al., supra; Bosher, J. M. and Labouesse, M. (2000) Nature Cell Biology 2, 31-36; Zamore, P. D., Tuschl, T., Sharp, P. A. and Bartel, D. P. (2000) Cell 101, 25-33; and Elbashir et. al. (2001) Nature 411, 494-498).

Briefly, PTGS/RNAi involves the intracellular defense system of the cell, which directs an RNA-dependent RNA polymerase (RdRp) or RNA-directed endoribonuclease (RDE) to generate many short interfering RNA fragments (si-RNAs) from the aberrant dsRNA or dsDNA template. The si-RNA can be further targeted by the RDE (or RNase III) for the fast degradation of its homologous or complementary gene transcripts (Scott W. Knight and Brenda L. Bass (2001) Science 293, 2269-2271). However, because a single strand RNA construct is highly susceptible to fast degradation and the RdRp/RDE is more sensitive to double-stranded templates, current scientists prefer to use double-stranded RNA (dsRNA; Fire, supra) as an aberrant template for better transfection results.

Although an RdRp-independent endoribonucleolysis model has also been proposed for the RNAi effect in Drosophila, the RdRp homologues were widely found in Arabidopsi thalianas as Sde-1/Sgs-2, in Neurospora crassa as Qde-1; and in Caenorhabditis elegans as Ego-1. Zamore, et al. supra; Yang, D., Lu, H., and Erickson, J. W. (2000) Current Biology 10, 1191-1200; Cogoni, C. and Macino, G. (1999) Nature 399, 166-169; Smardon, A., Spoerke, J. M., Stacey, S. C., Klein, M. E., Mackin, N., and Maine, E. M. (2000) Curr. Biol. 10, 169-171. Thus, RdRp homologues appear to be a prerequisite for maintaining a long-term/inheritable PTGS/RNAi effect (Bosher, et al. supra).

Although PTGS/RNAi phenomena appear to offer a potential avenue for inhibiting gene expression, they have not been demonstrated to work well in higher vertebrates and, therefore, their widespread use in higher vertebrates is still questionable. For example, all currently found RNAi effects are based on the use of double-stranded RNA (dsRNA), which have shown to cause interferon-induced non-specific RNA degradation (Stark et. al. (1998) Annu. Rev. Biochem. 67, 227-264; and Elbashir et. al. (2001) Nature 411, 494-498; U.S. Pat. No. 4,289,850 to Robinson; and U.S. Pat. No. 6,159,714 to Lau). Such interferon-induced cellular response usually reduces the specific gene silencing effects of RNAi phenomena and may cause cytotoxic killing effects to the transfected cells. In mammalian cells, it has been noted that dsRNA-mediated RNAi phenomena are repressed by the interferon-induced RNA degradation when the dsRNA size is larger than 30 base-pairs or its concentrations are more than 20 nM (Elbashir supra). For therapeutic use, the above limitations impair the usefulness of dsRNA because it would be difficult to deliver such small size and amount of dsRNA in vivo due to the high RNase activities of our bodies. Consequently, there remains a need for an effective and sustained method and composition for inhibiting gene function.

### SUMMARY OF THE PRESENT INVENTION

The present invention provides a novel composition and method for inhibiting gene function in higher eukaryotes in vivo. Without being bound by any particular theory, this method potentially is based on a posttranscriptional gene silencing phenomenon, potentially similar to PTGS/RNAi, which is hereafter termed DNA-RNA interference (D-RNAi). In accordance with the present invention DNA-RNA hybrids are used for inhibiting gene function. For example, both of the sense RNA (mRNA)-antisense DNA (cDNA) and the mismatched sense DNA (sDNA)-antisense RNA (aRNA) hybrids of the present invention have been shown to target a gene selected from the group consisting of pathogenic nucleic acids, viral genes, mutated genes, oncogenes and so on.

Alternatively, the present invention relating to DNA-RNA gene knockout technology can be used as a powerful new strategy in the field of gene medicine. The strength of this novel strategy is in its low dose, stability, and potential long-term effects. Applications of the present invention include, without limitation, the suppression of cancers by knocking out cancer-related genes, the prevention and treatment of microbe infections by knocking out microbe-related genes, the study of candidate molecular pathways with systematic knock out of involved genes, and the high throughput screening of gene functions based on transcript knocking out of large number of genes, possibly in conjunction with microarray or gene chip analysis, etc. The present invention can also be used as a tool for studying gene function in physiological conditions. The present invention provides a composition and method for altering the characteristic of a eukaryotic cell.

In specific embodiments, the present invention provides a method for gene silencing, comprising the steps of: a) providing: i) a substrate expressing a targeted gene, and ii) a composition comprising a DNA-RNA hybrid capable of silencing the expression of the targeted gene in the substrate; b) treating the substrate with the composition under conditions such that gene expression in the substrate is inhibited. The substrate can express the targeted gene in vitro or in vivo.

In one embodiment, the DNA-RNA hybrid targets a gene selected from the group consisting of pathogenic nucleic acids, viral genes, mutated genes, and oncogenes. In another embodiment, differently constructed DNA-RNA hybrids inhibit β-catenin oncogene expression in vitro and in vivo, while the sense RNA (sRNA)-antisense DNA (aDNA) hybrid inhibits bcl-2 expression in drug-resistant cancer cells. In yet another embodiment, both mRNA-cDNA (or termed sRNA-aDNA) and mismatched sDNA-aRNA hybrids suppress HIV-1 replication in CD4⁺ T cells from cell culture as well as patients' samples ex vivo.

The present invention provides a composition and method for altering the characteristic of a eukaryotic cell. In one aspect of the invention, the specific composition comprises a nucleic acid molecule that comprises a strand of deoxynucleic acid (DNA) molecule coupled to a strand of riboxynucleic acid (RNA) molecule. The RNA molecule is a sense RNA molecule that is homologous to a specific mRNA sequence to be targeted in the cell, while the DNA molecule is an antisense DNA molecule which is complemetary to the targeted mRNA, or vice versa. The RNA molecule also can be an antisense RNA molecule that is partially complementary to a specific messenger RNA (mRNA) sequence to be targeted in the cell, while the DNA molecule is a sense DNA molecule which is in the same orientation and contains homologous sequence composition as the targeted mRNA. The use of an RNA-DNA hybrid molecule is advantageous, among other reasons, because it does not trigger or otherwise has reduced occurrence of interferon-induced cytotoxicity, which is seen in the use of double-stranded RNA (dsRNA).

In yet another aspect of specific embodiments, the DNA and RNA strands of an RNA-DNA hybrid can be synthesized by either enzymatic or chemical reactions. The synthesized RNA and DNA are usually generated in the 5′ to 3′ direction (3′,5′-linkages) by polymerases; however, some chemical synthesizers do offer the 5′ to 2′ phosphodiester linkages (2′,5′-linkages), with or without hexose-containing nucleotide analog(s). Both 2′,5′-linked and 3′,5′-linked RNAs share a highly similar chemical and biological property, so as to the 2′,5′-linked and 3′,5′-linked DNAs (Hannoush et. al., (2001) J. Am. Chem. Soc. 123, 12368-12374). The synthesized RNA strand containing deoxynucleotide-structured backbone and/or modified nucleotide analog(s) can function as a DNA or RNA strand of the RNA-DNA hybrid molecule to protect the molecule from degradation and to increase knockout specificity. Therefore, the DNA and RNA strands of the DNA-RNA hybrid may comprise at least one nucleotide analog such as inosine, xanthine, hypoxanthine, deoxyuracil, ribonucleotide, labeled nucleotide, 7-deaza-dNTP, methylthio-linked nucleotide, phosphothio-linked nucleotide, morpholino nucleotide, peptide nucleic acid (PNA), viral genome nucleic acid and so on. In another embodiment, the percent complementation of the DNA and RNA molecules can range from about 20% to 100%, with the preferred range between about 50% and about 90%. The RNA molecule may be as long as the targeted mRNA molecule or may be shorter than the targeted mRNA molecule. The percent complementation between the aRNA and the targeted mRNA may range from about 1 to 100%, with the preferred range between about 50% to 100%. Furthermore, compositions comprising a plurality of the DNA-RNA hybrid and articles of manufacture, such as kits and therapeutic, diagnostic, prognostic, and research reagents, comprising the DNA-RNA hybrid molecule or a plurality of the DNA-RNA hybrid molecules are also contemplated as part of the invention.

In another aspect of the invention, the introduction of the DNA-RNA hybrid molecule into the eukaryotic cell is useful and shown to alter the characteristic of the eukaryotic cell. By the introduction of the DNA-RNA hybrid, the natural cellular defense mechanism, such as the RNA interference phenomena, may interfere and impair the expression of mRNA, which is homologous to or complementary to the RNA molecule of the DNA-RNA hybrid. Some examples of the characteristics of the cell that may be altered include: expression of a cellular protein; cell viability, cellular metabolism, cell division; expression of a viral RNA and/or protein, replication of a pathogen genome, and any other cellular functions that contribute to the characteristics of the cell. Examples of cellular protein include proteins expressed from genes such as oncogenes, cell cycle related genes, signal transduction pathway related genes, and any other genes in the cell. Alteration of cellular characteristics may be useful for therapeutic applications such as reducing the proliferation of cancer cells, reducing viral or pathogenic infection by reducing the expression of vital pathogenic genes in the cell, and any other applications where interfering with the expression of the RNA and/or protein in the cell would have a beneficial and therapeutic effect. Alteration of cellular characteristics may also be useful in the study of gene or protein function. By interfering or impairing the expression of the mRNA and/or protein, the protein function can be elucidated from the phenotype of the cell lacking the protein. Alteration of cellular characteristics may further be rendered by specific breakdown or degradation of non-gene-coding RNA molecules such as tRNA, introngenic RNA sequences, small RNA molecules, viral RNA genome, and other non-coding RNA molecules which may have known or unknown cellular functionality.

In another aspect of the invention, a method is provided for producing the DNA-RNA hybrid molecule, which is capable of altering the characteristic of a cell. Briefly, the method comprises the steps of synthesizing the RNA molecule, synthesizing the DNA molecule, and forming the DNA-RNA hybrid molecule from the RNA and DNA molecule. The RNA may be synthesized chemically, or through in vitro transcription from a double- or single-stranded nucleic acid template having a RNA polymerase promoter sequence or replicase recognition site. The DNA molecule may be synthesized chemically, by polymerase chain reaction (PCR), or through reverse transcription from an RNA molecule. The DNA-RNA hybrid molecule may also be formed by repeated steps of in vitro transcription and reverse transcription.

### BRIEF DESCRIPTION OF THE DRAWINGS

Referring particularly to the drawings for the purpose of illustration only and not limitation, there is illustrated:

FIG. 1 shows a schematic representation of the RNA-PCR method for DNA-RNA hybrid amplification.

FIG. 2 shows a schematic representation of experimental procedures for testing interference of bcl-2 gene expression in androgen-treated human prostate cancer LNCaP cells, according to one embodiment of the present invention.

FIG. 3 shows different templates for bcl-2 gene interference, according to one embodiment of the present invention.

FIG. 4 shows a proposed model for long-term PTGS/RNAi mechanisms.

FIG. 5 shows a linear plot of the interaction between incubation time and cell growth number in the methods of the present invention.

FIG. 6 shows potential D-RNAi-related RdRp enzymes by different α-amanitin sensitivity.

FIG. 7 shows a schematic representation for producing DNA-RNA hybrids.

FIG. 8 shows Northern results of blank control and sRNA-aDNA hybrid in one embodiment of the present invention.

FIG. 9 shows the effect of in vivo delivery of sRNA-aDNA hybrid on targeted gene expression, in one embodiment of the present invention. FIG. 9A shows an embryonic liver prior to microinjection; FIG. 9B shows the liver after injection. FIG. 9C shows the Northern analyses results after treatment with sRNA-aDNA hybrid, while FIG. 9D shows those of the liposome control embryos.

FIG. 10 illustrates the suppression of β-catenin, according to one embodiment of the present invention.

FIG. 11 is a general illustration of the preferred embodiment of mismatched DNA-RNA hybrids of the subject invention.

FIG. 12 is the in-cell results of example 12 of the subject invention.

FIG. 13 is the ex-vivo results of example 13 of the subject invention.

FIG. 14 is the in-vivo result of example 14 of the subject invention.

### DETAILED DESCRIPTION OF THE PREFERRED EMBODIMENT

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

Gene Silencing Using RNA-DNA Hybrids: In Vitro Prostrate Cancer Model

As noted earlier, posttranscriptional gene silencing (PTGS) and RNA interference (RNAi) have been found capable of quelling specific gene activities in a variety of in vivo systems.

According to the invention provided herein, ectopic transfection of a sequence-specific RNA-DNA hybrid (instead of a transgene or ds-RNA) is used to induce intracellular gene silencing in human cells. Although previous transgene/ds-RNA transfection experiments showed that PTGS/RNAi effects are limited to plants and some simple animals, using the present invention, specific gene interference of bcl-2 expression in human LNCaP prostate cancer cells using the long RNA-DNA hybrid has been successfully detected.

Normal human prostatic secretory epithelial cells do not express bcl-2 protein, whereas neoplastic prostate tissues from androgen-ablation patients show an elevated level of this apoptosis-suppressing oncoprotein. It is known in the art that over-expression of bcl-2 protects prostate cancer cells from apoptosis in vitro, and confers resistance to androgen depletion in vivo. The tumorigenic and metastatic potentials of LNCaP cells are also significantly increased after bcl-2 stimulation by either androgen or transgene treatment. Such inhibition of apoptosis can be blocked by treatment with bcl-2 antisense oligonucleotides, but many apoptotic stimuli such as etoposide or phorbol ester cannot be blocked.

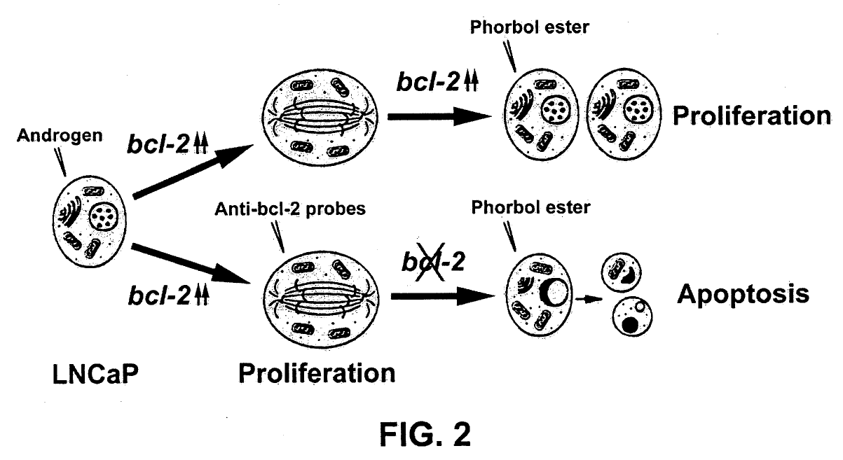

The potential utility of RNA-DNA hybrid in preventing bcl-2 expression was therefore tested on androgen-stimulated LNCaP cells, expecting to increase cancer cell susceptibility to apoptotic stimuli and reduce tumorigenic outgrowth in vitro. Following previous findings, LNCaP cells were treated with dihydrotestersterone (100 nM 5α-anrostan-17β-3-one) to block the apoptotic effect of phorbol ester (10 nM phorbol-12-myristate-13-acetate). When treated with the methods and compositions of this invention LNCaP cells induced a bcl-2 knockdown effect to resume the apoptosis of the androgen- and phorbol ester-treated cancer cells (FIG. 2).

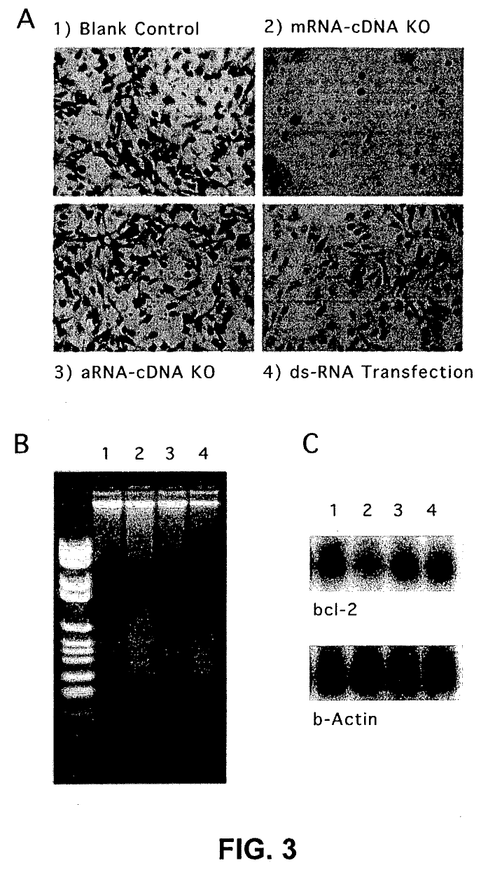

FIG. 3 shows the analysis of different templates for bcl-2 gene interference, namely: (1) blank control; (2) mRNA-cDNA hybrid (the same as sRNA-aDNA hybrid); (3) perfectly matched aRNA-cDNA hybrid; and (4) dsRNA in LNCaP cells. FIG. 3A shows changes of cell proliferation rate and morphology. Chromosomal DNAs were stained by propidium iodide. Although the dsRNA transfection also showed minor morphological changes, a significant cell growth inhibition and chromosomal condensation only occurred in the mRNA-cDNA transfection (n=4). FIG. 3B shows genomic laddering patterns demonstrating apoptosis induction by the bcl-2 mRNA-cDNA transfection. FIG. 3C presents Northern blots showing a strong gene silencing effect of the mRNA-cDNA transfection in bcl-2 expression. As shown in FIG. 3, the transfection of bcl-2 mRNA-cDNA hybrids (5 nM) into LNCaP cells was sufficient to silence bcl-2 expression and cause apoptosis (chromosomal condensation and genomic DNA laddering fragmentation), which have not been found in else.

There are three major effects of PTGS, i.e., initiation, spreading and maintenance, all of which are also found in many inheritable RNAi phenomena. The initiation indicates that the onset of PTGS/RNAi takes a relatively long period of time (13 days) to develop enough small RNAs or short aRNAs for specific gene knockout. With the antisense transfection processes, it only takes several hours to reach the same gene silencing results but with much higher dosages and higher cytotoxicity. Also, unlike the short-term effectiveness of traditional antisense transfections, the PTGS/RNAi effects may spread from a transfected cell to neighboring cells and can be maintained for a very long time (weeks to lifetime) in a mother cell as well as its daughter cells.

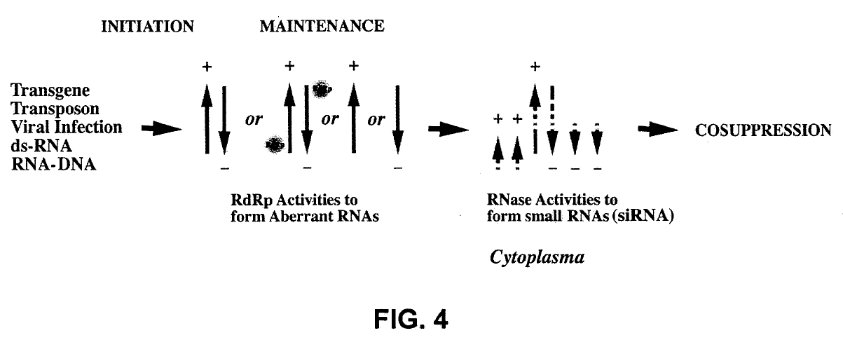

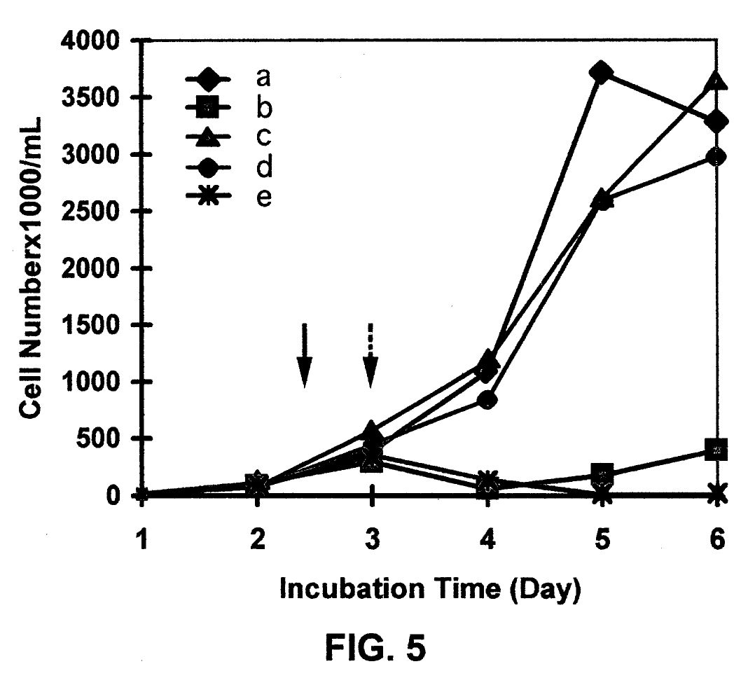

The results of the experiments here suggest that the invention shares some features of the PTGS/RNAi mechanisms. FIG. 4 shows a proposed model for long-term PTGS/RNAi mechanisms. Initiation and maintenance periods are varied, depending on different living systems and transfected genes. Because liposomal transfection methods offer only 30˜40% transfection rate, a complete apoptosis induction in the LNCaP cell model used required at least two to three transfections (FIG. 5). FIG. 5 shows a linear plot of the interaction between incubation time (X) and cell growth number (Y), indicating no spreading effect. The black linear arrow shows the first addition of all tested probes, while the dotted arrow indicates the second addition of an mRNA-cDNA probe for double transfection analysis. The growth of mRNA-cDNA (red and black) transfected cells remarkably inhibited after 36-hour incubation (n=4). Because one transfection is not sufficient to reach the entire cell population, a more complete inhibition of cell growth is achieved after double transfections (black), indicating no or rare spreading effect.

Identification of A Potential RdRp-Like Enzyme for Gene Silencing in LNCaP Cells

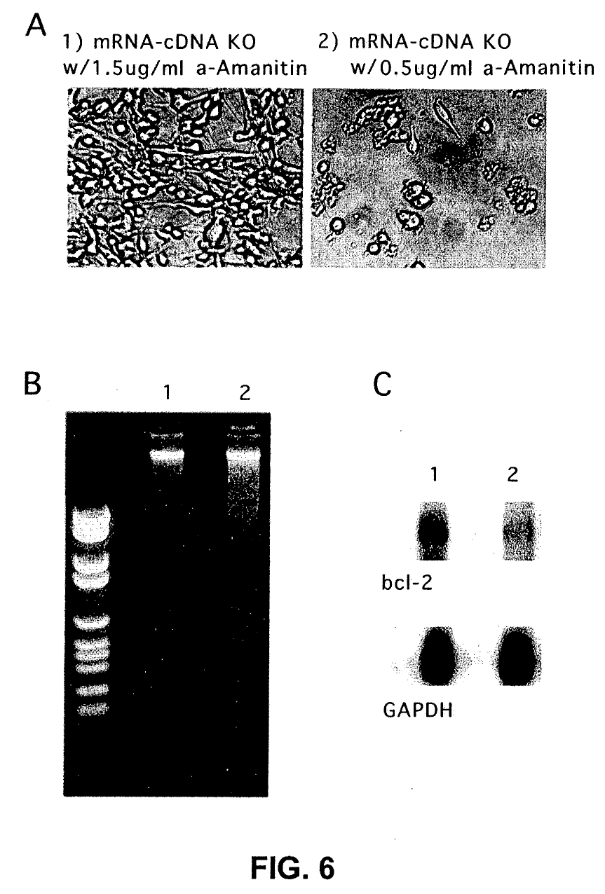

RNA polymerase II has been found to possess RNA-directed RNA synthesis activity (Filipovska et al., RNA 6: 41054 (2000); Modahl et al., Mol. Cell Biol. 20: 6030-6039 (2000)). Furthermore, the addition of low-dose α-amanitin (1.5 μg/ml), an RNA polymerase II-specific inhibitor derived from a mushroom Amanita phalloides toxin, abrogated the apoptosis induction of bcl-2 D-RNAi (FIG. 6).

FIG. 6 shows an analysis of a potential RdRp enzyme by different α-amanitin sensitivity: (1) 1.5 μg/ml and (2) 0.5 μg/ml. FIG. 6A shows the changes of cell proliferation rate and morphology after addition of α-amanitin. A significant reduction of apoptosis was detected in the 1.5 μg/ml α-amanitin addition (but not in the 0.5 μg/ml α-amanitin addition) after mRNA-cDNA (equal to sRNA-aDNA) transfection (n=3), showing a dose-dependent release of cell growth inhibition FIG. 6B shows genomic laddering patterns demonstrating the blocking of the apoptotic induction effect of the bcl-2 mRNA-cDNA transfection by the 1.5 μg/ml α-amanitin addition. FIG. 6C shows Northern blots indicating that the bcl-2 silencing effect was prevented.

Gene Silencing Using RNA-DNA Hybrids: In Vivo Model Targeting β-Catenin in Developing Chicken Embryos

As shown in the form of mRNA-cDNA duplex above, the foregoing establishes that the novel sRNA-aDNA hybrids of the present invention can be used in a novel strategy to knock out targeted gene expression in vitro. As discussed below, the novel sRNA-aDNA strategy of the invention is also effective in knocking out gene expression in vivo.

As illustrated in the examples below, the methods and compositions of the invention are effective in knocking out targeted gene expression in vivo in a developing chicken embryo. For molecules, α-catenin was targeted because it has a critical role in development and oncogenesis, and for tissue, skin and liver were selected because the skin is accessible and the liver is an important organ. β-catenin is known to be involved in the regulation of growth control. It has been suggested that β-catenin is involved in neovasculogenesis and that it may work with VE-cadherin, which is not essential for the initial endothelial adhesion but is required in further vascular morphogenesis to properly form mature endothelial walls and blood vessels.

As discussed above, the experimental results establish that sRNA-aDNA (as shown in mRNA-cDNA) hybrids potently inhibit β-catenin expression in the liver and skin of developing chick embryos. Thus, the results show that using a sRNA-aDNA duplex provides a powerful new strategy for gene silencing. A perfect matched sDNA-aRNA duplex usually does not appear to work well even though dominant-negatively transfected aRNA has been previously shown to suppress gene expression. This is due to the strong affinity of the completely matched sDNA-aRNA hybrid which exclude the engagement of RDE activity needed for RNAi onset. The results also show that this invention is effective in knocking out the targeted gene expression over a long period of time (>10 days). Further, it was observed that non-targeted organs appear to be normal, which implies that the compositions herein possess no overt toxicity. Thus, the invention offers the advantages of low dosage, stability, long term effectiveness, and lack of overt toxicity.

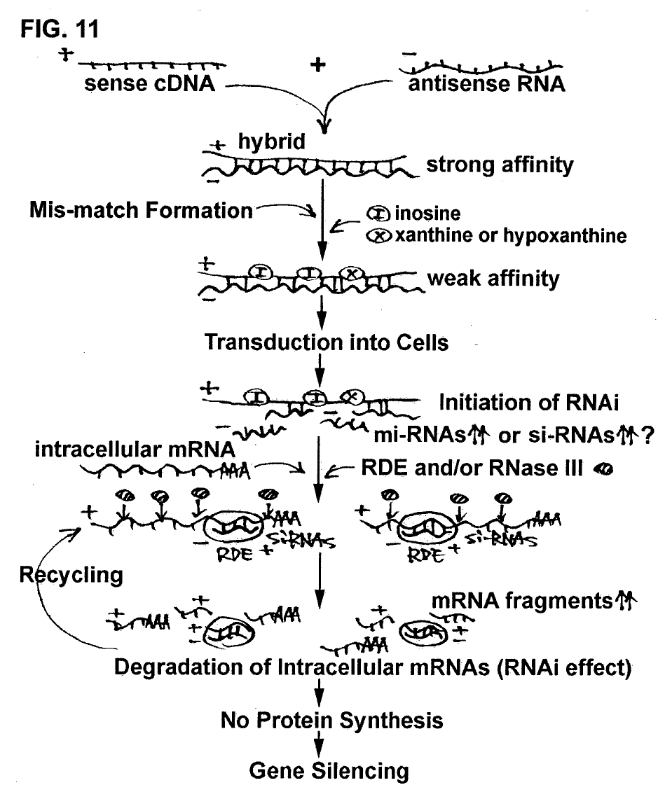

By disrupting the matched sequence(s) of a DNA-RNA hybrid, the present invention also provides a novel composition and method for altering a characteristic of a cell. Without being bound by any particular theory, the alteration of a characteristic of the cell may be based on an RNAi-dependent gene silencing phenomenon, triggered by the introduction of a DNA-RNA hybrid molecule into the cell. Generally, as seen in FIG. 11, when the DNA-RNA hybrid molecule is transduced, transfected, or otherwise introduced into the cell, small fragments of si-RNAs may be produced by cleavage of the RNA or disassociation of the RNA from the DNA strand. The si-RNAs hybridize to the targeted mRNA present in the cells and the mRNA becomes targets for degradation by RDE and/or RNase III present in the cell. Because the targeted mRNA molecules are degraded, no protein synthesis occurs resulting in the silencing of the gene from which the mRNA was transcribed.

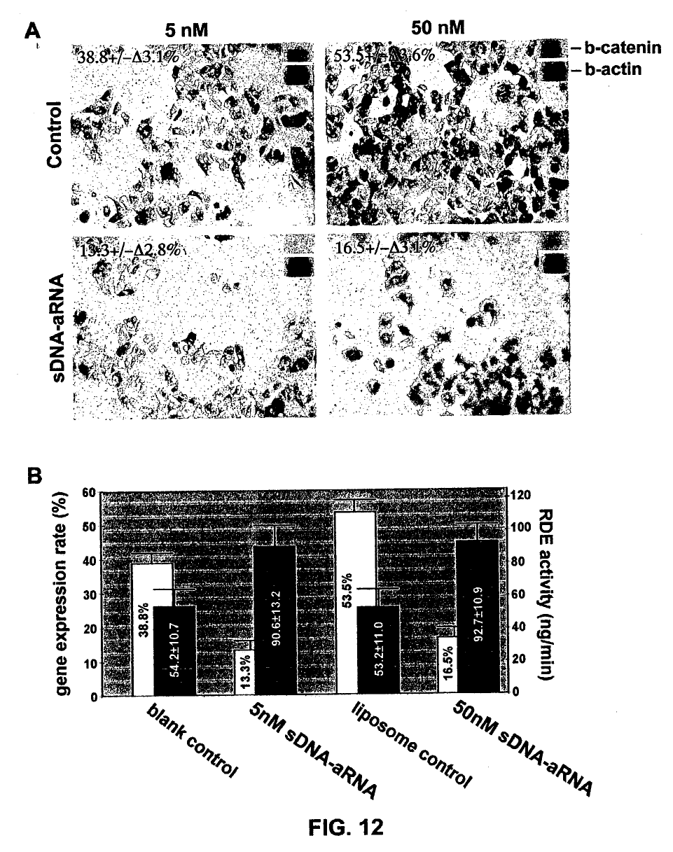

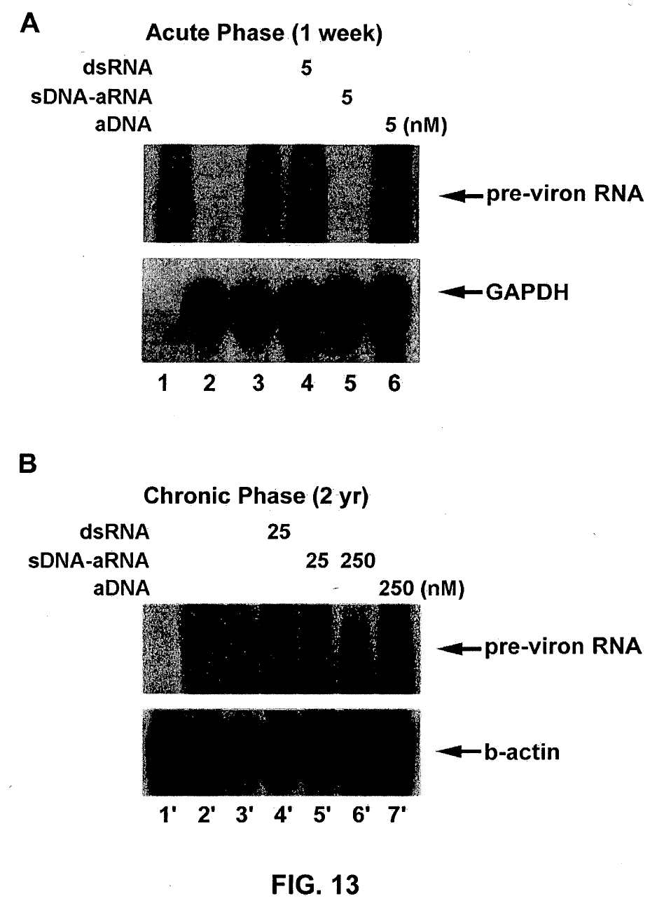

Some of the advantages of the DNA-RNA hybrid molecule over dsRNA transfection are listed as follows: 1) the DNA portion of a DNA-RNA hybrid can be modified to stabilize the efficacy of RNAi phenomenon induction; 2) the RNA portion of a DNA-RNA hybrid is well protected by the DNA portion of the same for more stable transfection (Lin (2001) supra); 3) the RNAi-associated RNA-directed endoribonuclease has experimentally been shown to possess high activity to the RNA portion of a DNA-RNA hybrid (see FIG. 12(b)); 4) the DNA-RNA hybrid construct has been tested to suppress the interferon-induced cytotoxicity which is usually caused by dsRNA (see FIG. 13(b) and Example 13); and 5) the size of a DNA-RNA hybrid can be larger than 30 base pairs for more effective transfection and specific gene targeting.

As seen in FIG. 11, the RNA of the DNA-RNA molecule is an RNA complementary to the targeted messenger RNA (mRNA) in the cell. The RNA is coupled to a DNA which is homologous to the sequence of the targeted mRNA in the cell. The complementation between the DNA molecule and the RNA molecule of the DNA-RNA hybrid may range from about 20% to 100%, with the preferred range between about 45% to about 99%, most preferably about 95% in a linearly complementary form or about 48% in a palindromic form sequence. One way of introducing mismatch and therefore reduce the affinity of the DNA molecule and the RNA molecule is to treat the hybrid with enzymes or chemicals that will generate nucleotide analogs in the DNA strand of the DNA-RNA hybrid. For example, the nucleotide analogs can be generated by adding deaminase or acidic chemicals to the DNA portion of the DNA-RNA hybrid, resulting in analogs such as of inosine (I), xanthine (x), hypoxanthine (HX), uracil (U), DNA-linked ribonucleotides and their derivative analogs (See e.g., U.S. Pat. No. 6,130,040, which is hereby incorporated by reference). Other methods of generating deoxynucleotide analogs include direct incorporation of analogs (inosine (I), xanthine (x), hypoxanthine (HX), deoxyuracil (dU), ribonucleotide in a DNA linkage, deoxyribonucleotide in an RNA linkage, 7-deaza-dNTP, labeled nucleotides, and their derivative analogs, such as hexose-containing, 2′-5′ linked, phosphothio-linked, methylthio-linked, morpholino-linked and peptide-linked nucleotide analogs) during the synthesis of the DNA and/or RNA. The DNA can be synthesized chemically by using an oligonucleotide synthesizer or through in vitro enzymatic reactions such as PCR, reverse transcription, DNA polymerase extension reaction wherein the deoxynucleotide and deoxynucleotide analogs are present in the reaction.

In accordance with one aspect of the present invention, DNA-RNA hybrids are used for inhibiting gene function. For example, the DNA-RNA hybrid gene knockout or gene silencing technology can be used as a powerful new strategy in the field of gene-based therapy. As seen in the examples discussed below, the advantages of this novel strategy are in its low dose, stability, and potential long-term effects. The DNA-RNA hybrids of the present invention can be used to target a gene such as functional genes, pathogenic nucleic acids, viral genes/genomes, bacterial genes, mutated genes, oncogenes and any other genes functionally expressing RNA or protein. Examples of oncogenes are β-catenin, bcl-2, c-myc, etc. Examples of functional genes include tyrosinase, p53, TNF-α, etc. Examples of virus, the genes of which can be targeted, include HIV, HCV, Rhinovirus, Herpes virus, Papilloma virus, CMV, Ebola, and any other pathogenic or oncogenic viruses.

Although the preferred target for RNAi is the mRNA in the cell, not all targeted RNA for RNAi degradation are required to be capable of expressing a protein. Other types of RNA such as t-RNA, r-RNA, present naturally in the cells may also be targeted if desired. Furthermore, non-protein expressing portion of RNA viruses, for example, which replicate in eukaryotic cells may also be targeted for degradation, thereby reducing the ability of the virus to replicate.

The inhibition of gene expression by the DNA-RNA hybrid molecule may also be applied to study the gene function of unknown nucleic acid transcripts. For example, DNA-RNA hybrid molecule may be generated where the DNA is homologous to the gene or mRNA with an unknown function. The inhibition of the expression of a protein from the mRNA may then alter the characteristic of the cell, which would provide clues as to the possible function of the gene. Candidate genes for study may be identified by differential expression of the gene in two different types of cells (e.g., cancerous cells vs. non-cancerous cells; muscle vs. brain) or at different stages of cell cycle or animal development. Such identification by differential expression may be achieved using subtractive hybridization, differential display, array or microarray technologies, and any other techniques used for comparing the gene expression in two different cells.

To increase the onset of RNAi-related effects in cells, the DNA portion of a DNA-RNA hybrid can be modified to increase the efficiency of release of the RNA portion to a RNAi-associated RNA-directed endoribonuclease (RDE). Such modification can be accomplished either by the incorporation of weak binding nucleotide analogs during the synthesis of the DNA portion or the deamination of DNA sequence nucleotides after its synthesis. For the incorporation method, the nucleotide analogs are integrated into the DNA sequence using an oligonucleotide synthesizer machine (e.g. SEQ ID.19) or an enzymatic reaction, such as reverse transcription (RT), polymerase chain reaction (PCR), nucleic acid sequence based amplification (NASBA) and RNA-polymerase cycling reaction (RNA-PCR) (e.g. Example 12). The nucleotide analog can be selected from the group consisting of ribonucleotide in DNA linkage, deoxyuracil (dU), inosine (I), xanthine (X), hypoxanthine (HX) and their derivative analogs, such as hexose-containing, 2′-5′ linked, phosphothio-linked, methylthio-linked, morpholino-linked and peptide-linked nucleotide analogs. Alternatively, the nucleotide analog can be generated by adding deaminase or acidic chemicals (e.g. acetic acid) to the DNA sequence, resulting in derivative analog(s) selected from the group consisting of inosine (I) and its derivatives (See e.g., U.S. Pat. No. 6,130,040).

Furthermore, the percent complementation between the DNA molecule and the RNA molecule in the DNA-RNA hybrid may be adjusted, resulting in stronger or weaker hybridization between the two strands. The percent complementation may range from about 20% to 100%, whereby the percent complementation is determined by the ratio of the mismatching bases between the DNA molecule and the RNA molecule and the number of total bases (matched+mismatched) in one strand of the molecule (DNA or RNA). For example, if a particular DNA-RNA hybrid molecule has the particular sequence as shown below, the percent complementation is 75% (1 mismatch G/U to 4 total bases):

| | |
|---|---|
| DNA | G G C G |
| RNA | C U G C |

Preferably, the percent complementation between the DNA and the RNA molecule in the DNA-RNA hybrid molecule is between about 50% to about 90%.

The length of the DNA-RNA hybrid molecule can also be controlled and adjusted if desired. Unlike dsRNA, the DNA-RNA hybrid molecule does not trigger interferon-induced cytotoxicity in the targeted cells. Thus, the DNA-RNA hybrid may range from as small as 20 basepairs to 10 kilobasepairs, preferably ranging from 50 basepairs to 500 basepairs, most preferably, in the range of 75 basepairs to 150 basepairs.

Method of Generating the DNA-RNA Hybrid.

The DNA-RNA hybrid may be generated in a number of different ways. The DNA and the RNA may, for example, be separately synthesized by chemical synthesis using an oligonucleotide synthesizer. After synthesis, the DNA and the RNA strands may then be combined together by allowing them to anneal to each other. Alternatively, the DNA may also be synthesized from the chemically synthesized RNA using a reverse transcriptase enzyme, primers complementary to the RNA, and deoxynucleotides in a reverse transcription reaction.

The RNA may also be synthesized through in vitro transcription of the RNA from a double stranded DNA that has an RNA polymerase sequence such as T7, SP6 or T3 RNA polymerase promoter. The double stranded DNA may be a plasmid, or a linear piece of DNA generated by PCR or restriction digest. After the in vitro transcription reaction, the resulting RNA may then be the source of template for a reverse transcriptase reaction to generate DNA-RNA hybrid molecules. Resulting DNA-RNA hybrid molecules from the reverse transcription reaction may be purified before use or may be used directly without purification. Examples of purification of the DNA-RNA hybrid molecules after reverse transcription reaction include ethanol precipitation, column chromatography and gel filtration.

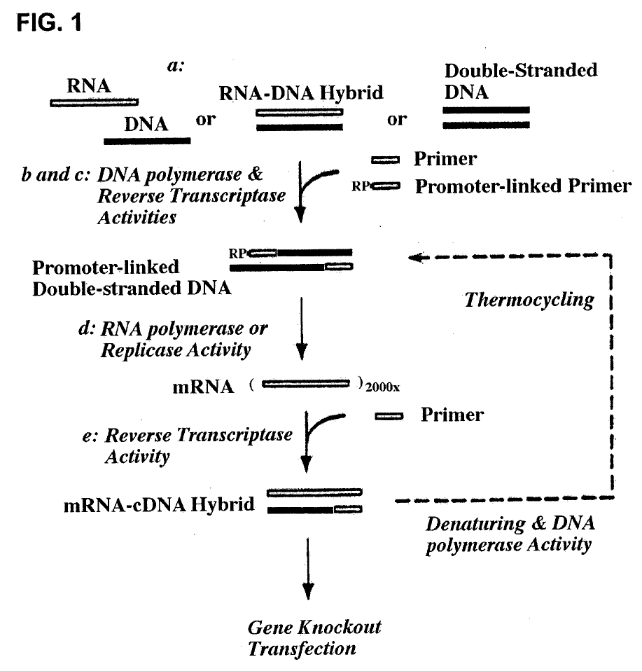

Preferably, the DNA-RNA hybrid may be generated by an improvement of the so-called RNA-PCR described in U.S. Pat. No. 6,197,554 by common inventors in this application. As seen in FIG. 1, the starting material for generating the DNA-RNA hybrid molecule can be any type of nucleic acid molecule such as single stranded DNA or RNA, double stranded DNA, or DNA-RNA hybrids. The modification of the RNA-PCR method disclosed in the '554 patent involves the use of a gene-specific primer and promoter-primer in the thermocycling procedure to amplify specific DNA-RNA sequences for gene knockout technologies. This thermocycling procedure preferably starts from reverse transcription of mRNAs with RNA promoter-containing primer(s) and Tth-like polymerases, followed by DNA double-stranding reaction with the same Tth-like polymerases. The resulting promoter-linked double-stranded DNAs may then serve as transcriptional templates for amplifying RNA amount up to 2000 fold/cycle by RNA polymerases. The thermocycling procedure can be repeated for more amplification of the DNA-RNA hybrids. (Again, as seen in FIG. 4, the starting material can be from double stranded DNA, which would mean that instead of starting with a reverse transcriptase reaction, the double stranded DNA are denatured, annealed with the promoter linked primer, and extended using DNA polymerases such as Taq DNA polymerase to generate a double stranded DNA having a RNA polymerase promoter sequence.)

The amplification cycling procedure of the present invention presents several advantages over prior amplification methods. First, DNA-RNA probes from low-copy rare mRNA species can be prepared within three round of amplification cycling without mis-reading mistakes. Second, the DNA-RNA hybrid amplification is linear and does not result in preferential amplification of nonspecific gene sequences. Third, the RNA degradation is inhibited by thermostable enzymatic conditions with RNase inhibitors. Finally, the use RNase H negative reverse transcriptase which reduce the activity of RNase H preserves the integrity of final DNA-RNA constructs. Unlike previous NASBA methods (Compton, Nature 350: 91-92 (1991)), this improved RNA-PCR procedure contains no RNase H activity which usually destroys the RNA structure of a DNA-RNA hybrid. Based on these advantages, high amount of pure and specific DNA-RNA hybrids can be prepared for transducing biological effects of interest in vitro, ex vivo as well as in vivo.

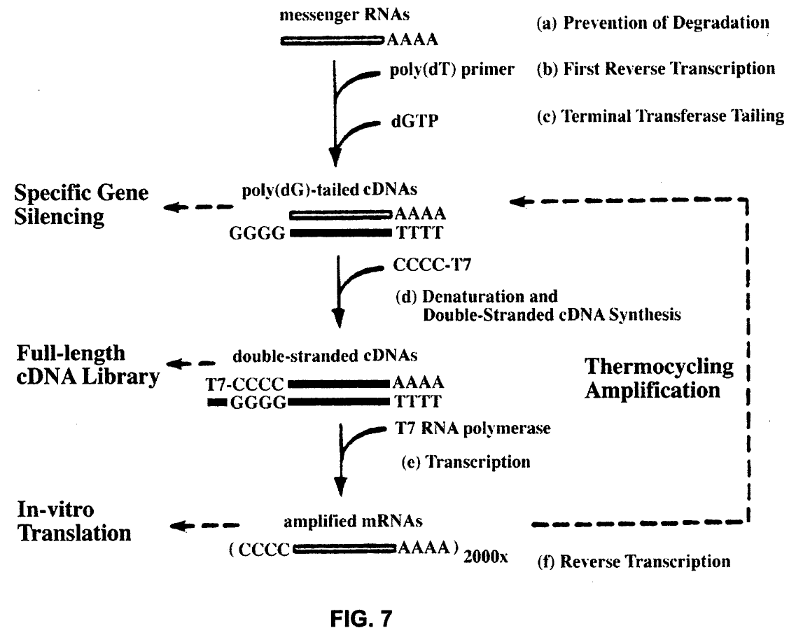

Another advantage of the preferred method, as exemplified in FIG. 7, is that labor and time are reduced because of the high amplification efficiency of RNA polymerase (up to 2000 folds/cycle). Also, such preparation of amplified DNA-RNA hybrids is less expensive and more efficient than traditional cDNA cloning with an expression-competent plasmid vector and then reverse transcription of the expressed RNA products. Most importantly, this DNA-RNA hybrid amplification can be carried out in a microtube with only a few nucleic acid template (0.2 pg). Taken together, these special features make the improved content of RNA-PCR as simple, fast, and inexpensive as a kit for concisely isolating amplified DNA-RNA hybrid sequences for specific gene knockout assays.

Although certain preferred embodiments of the present invention have been described, the spirit and scope of the invention is by no means restricted to what is described above. For example, within the general framework of: a) one or more types of nucleic acid templates used; b) one or more specific primers for reverse transcription and polymerase extension reactions; c) one or more promoter-linked primers for transcription reactions; d) one or more enzymes for each step of reaction(s); e) one or more rounds of the cycling procedure for DNA-RNA hybrid amplification, there is a very large number of permutations and combinations possible, all of which are within the scope of the present invention.

In yet another example, the DNA-RNA hybrid can be generated by a method comprising: a) providing: i) a solution comprising a nucleic acid template, ii) one or more primers sufficiently complementary to the sense conformation of the nucleic acid template, and iii) one or more promoter-linked primers sufficiently complementary to the antisense conformation of the nucleic acid template, and having an RNA promoter; b) treating the nucleic acid template with one or more primers under conditions such that a first DNA strand is synthesized; c) treating the first DNA strand with one or more promoter-linked primers under conditions such that a promoter-linked double-stranded nucleic acid is synthesized; d) treating the promoter-linked double-stranded nucleic acid under conditions such that essentially RNA fragments are synthesized; and e) treating RNA fragments with one or more primers under conditions such that a DNA-RNA hybrids are synthesized. The steps of (b) through (e) may also be repeated for a sufficient number of cycles to obtain a desired amount of amplified hybrid product.

The treating step in step (b) can also comprise heating the solution at a temperature above 90° C. to provide denatured nucleic acids. The treating step in step (c) can also comprise treating the first DNA strand with one or more promoter-linked primers at a temperature ranging from about 37° C. to about 70° C., depending on the annealing sequence region used. The treating step in step (c) can also comprise treating the DNA strand with one or more promoter-linked primers in the presence of a polymerase.

In one embodiment, the polymerase used in the above methods may include DNA-dependent DNA polymerases, RNA-dependent DNA polymerases, RNA polymerases, Taq-like DNA polymerase, Tth-like DNA polymerase, C. therm. polymerase, viral replicases, and combinations thereof. The viral replicases may include avian myeloblastosis reverse transcriptase, Moloney murine leukemia virus reverse transcriptase, and their derivatives that do not have RNase H activity, and Brome mosaic virus replicase, Trichomonas vaginalis virus replicase, Flock house virus replicase, Q beta replicase, and their mutants and/or combinations thereof.

The treating step in step (d) can also comprise treating the promoter-linked double-stranded nucleic acid with an enzyme having transcriptase activity at about 37° C. The enzyme having transcriptase activity can be selected from the group consisting of RNA polymerases and viral replicases. The RNA polymerases can be selected from the group consisting of T3 RNA polymerase, T7 RNA polymerase, SP6 RNA polymerase, and M13 RNA polymerase, Brome mosaic virus replicase, Trichomonas vaginalis virus replicase, Flock house virus replicase, Q beta replicase, and their mutants and/or combinations thereof.

As to the primers, the primers may be complementary to the 3′-ends of the antisense conformation of the nucleic acid template. In one embodiment, one or more primers comprise a sequence-specific primer homologous or complementary to the targeted gene transcript. The promoter-linked primers may include a sequence complementary to the 3′-ends of the sense conformation of the nucleic acid template and a sequence corresponding to the sequence of an RNA polymerase promoter. In one embodiment, one or more promoter-linked primers comprise a sequence-specific primer complementary to the targeted gene transcript, such as T7 promoter-linked poly(dT) primers. The promoter-linked double-stranded nucleic acid template can also include linear and circular promoter-containing double-stranded DNAs or promoter-linked single-stranded DNAs.

In one embodiment, the treating step in step (e) may comprise treating RNA fragments with one or more primers at a temperature ranging from about 35° C. to about 72° C., depending on the annealing sequence region used.

The synthesis of the DNA-RNA hybrid molecule, in accordance with any of the above methods, may also include a step of incorporating one or more nucleotide analogs into the DNA or RNA portion of the DNA-RNA hybrid to facilitate and increase the induction and onset of RNAi-related effects. The nucleotide analog is incorporated by either a chemical synthesizer or enzymatic reactions, or both. The nucleotide analog can be selected from the group consisting of ribonucleotide in the DNA construct, deoxynucleotide in the RNA construct, deoxyuracil (dU), inosine (I), xanthine (X), hypoxanthine (HX) and/or their derivative analogs in the hybrid construct. Alternatively, the nucleotide analog can be generated by adding deaminase or acidic chemicals to the DNA-RNA hybrid, resulting in derivatives selected from the group consisting of inosine (I) and its derivative analogs (See e.g., U.S. Pat. No. 6,130,040, which is hereby incorporated by reference).

Labeling of DNA-RNA hybrids may also be achieved by incorporation of labeled nucleotides or analogs during the reverse transcription of RNAs. The nucleotide sequences so generated are useful for tracking down the transfected cells in a large cell population. These labeled nucleotides are also capable of being probes in a variety of applications, such as Southern blots, dot hybridization, position cloning, nucleotide sequence detection, gene knockout transfection and so on. The incorporated nucleotide analogs also provide better protection of the DNA-RNA structures, resulting in more stability and effectiveness of the probe transfection. The nucleotide analog can be selected from the group consisting of biotin-labeled, digoxigenin-labeled, fluorescein-labeled, amino-methylcoumarin-labeled, tetramethyl-rhodamine-labed nucleotides and their derivatives.

Definitions

To facilitate understanding of the invention, a number of terms are defined below.

As used herein, the term “isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein. If the “isolated” composition occurs as an intermediate composition in an in vitro process, “isolated” composition does not mean the intermediate composition but the desired end product intended to be introduced into a cell. For example, an intermediate DNA-RNA hybrid that forms in an in-vitro transcription reaction is an intermediate and would not be “isolated.” On the other hand, the end product of a reverse transcriptase reaction from an RNA creating a DNA-RNA hybrid would be “isolated” even without purification of the hybrid from the enzymes, templates, nucleotides, primers, and buffers present in the reaction.

As used herein, the terms “complementary” or “complementarity” or “complementation” are used in reference to polynucleotides (i.e., a sequence of nucleotides) related by the base-pairing rules. For example, the sequence “A-G-T” is complementary to the sequence “T-C-A,” and also to “T-C-U.” Complementation can be between two DNA strands, a DNA and an RNA strand, or an RNA and another RNA strand. Complementarity may be “partial” or “complete” or “total”. Partial complementarity or complementation occurs when only some of the nucleic acid bases are matched according to the base pairing rules. Complete or total complementarity or complementation occurs when the bases are completely matched between the nucleic acid strands. The degree of complementarity between nucleic acid strands has significant effects on the efficiency and strength of hybridization between nucleic acid strands. This is of particular importance in amplification reactions, as well as in detection methods which depend upon binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

As used herein, the term “homologous” or “homology” refers to a polynucleotide sequence having similarities with a mRNA sequence or naturally occurring RNA sequence such as t-RNA, rRNA or RNA genome of an RNA virus. A nucleic acid sequence may be partially or completely homologous to a particular mRNA sequence, for example. Homology may also be expressed in percentage as determined by the number of similar nucleotides over the total number of nucleotides.

As used herein, the term “sDNA” refers to a single stranded DNA that is homologous to a mRNA sequence, while the term “sRNA” refers to a single stranded RNA that is the same as or homologous to a mRNA sequence. The term “aDNA” and “cDNA” refers to a single stranded DNA that is complementary to a mRNA sequence, while the term “aRNA” refers to a single stranded RNA that is complementary to a mRNA sequence.

As used herein, the term “sense conformation” refers to a nucleic acid sequence in the same sequence order and composition as its homolog mRNA. The sense conformation is indicated as a “+” symbol, or with a “s” in front of the DNA or RNA, e.g., “sDNA” or “sRNA.”.

As used herein, the term “antisense” refers to a nucleic acid sequence complementary to its respective mRNA molecule or a naturally occurring RNA molecule such as t-RNA, rRNA, or viral RNA. The viral RNA may be the genome of an RNA virus and may or may not encode for a functional protein. For example, the antisense RNA (aRNA) may refer to a ribonucleotide sequence complementary to a mRNA sequence, encoding for a protein, in an A-U and C-G composition, and also in the reverse orientation of the mRNA. The antisense conformation is indicated as a “−” symbol or with a “a” in front of the DNA or RNA, e.g., “aDNA” or “aRNA.”

As used herein, the term “template” refers to a nucleic acid molecule being copied by a nucleic acid polymerase or a chemical synthesizer. A template can be single-stranded, double-stranded or partially double-stranded, depending on the polymerase or chemical reaction. The synthesized copy is complementary to the template, or to at least one strand of a double-stranded or partially double-stranded template. Both RNA and DNA are usually synthesized in the 5′ to 3′ direction (3′,5′-linkages); however, some chemical synthesizers do provide the 5′ to 2′ phosphodiester linkages (2′,5′-linkages). The 2′,5′-linked and 3′,5′-linked RNA/DNA share the same functional properties to the purpose of the present invention. The two strands of a nucleic acid duplex are always aligned so that the 5′ ends of the two strands are at opposite ends of the duplex (and, by necessity, so then are the 3′ and/or 2′ ends).

As used herein, the term “nucleic acid template” refers to a double-stranded DNA molecule, double stranded RNA molecule, hybrid molecules such as DNA-RNA or RNA-DNA hybrid, or single-stranded DNA or RNA molecule.

As used herein, the term “palindromic sequence” refers to a segment of single- or double-stranded nucleic acid sequence in which the base sequence(s) of the strand duplex exhibit about twofold rotational symmetry about an axis. For example, the duplex of “TTAGCAC GTGCTAA” and “AATCGTG CACGATT”.

As used herein, the term “primer” refers to an oligonucleotide complementary to a template. The primer complexes with the template to give a primer/template complex for initiation of synthesis by a DNA polymerase. The primer/template complex is extended by the addition of covalently bonded bases linked at its 3′ end, which are complementary to the template in DNA synthesis. The result is a primer extension product. Virtually all known DNA polymerases (including reverse transcriptases) require complexing of an oligonucleotide to a single-stranded template (“priming”) to initiate DNA synthesis.

As used herein, the term “promoter-linked primer” refers to an RNA-polymerase-promoter sense sequence coupled with a gene-specific complementary sequence in its 3′-end for annealing to the antisense conformation of a nucleic acid template.

As used herein, the term “DNA-dependent DNA polymerase” refers to an enzyme that synthesizes a complementary DNA copy from a DNA template. Examples are DNA polymerase I from E. coli and bacteriophage T7 DNA polymerase. Under suitable conditions a DNA-dependent DNA polymerase may synthesize a complementary DNA copy from an RNA template.

As used herein, the terms “DNA-dependent RNA polymerase” and “transcriptase” refer to enzymes that synthesize multiple RNA copies from a double-stranded or partially-double stranded DNA molecule having a promoter sequence. Examples of transcriptases include, but are not limited to, DNA-dependent RNA polymerase from E. coli and bacteriophages T7, T3, and SP6.

As used herein, the terms “RNA-dependent DNA polymerase” and “reverse transcriptase” refer to enzymes that synthesize a complementary DNA copy from an RNA template. All known reverse transcriptases also have the ability to make a complementary DNA copy from a DNA template. Thus, reverse transcriptases are both RNA-dependent and DNA-dependent DNA polymerases. As used herein, the term “RNase H” refers to an enzyme that degrades the RNA portion of an RNA/DNA duplex. RNase H's may be endonucleases or exonucleases. Most reverse transcriptase enzymes normally contain an RNase H activity in addition to their polymerase activity. However, other sources of the RNase H are available without an associated polymerase activity. The degradation may result in separation of RNA from a RNA/DNA complex. Alternatively, the RNase H may simply cut the RNA at various locations such that portions of the RNA melt off or permit enzymes to unwind portions of the RNA.

As used herein, the terms “hybridize” and “hybridization” refer to the formation of complexes between nucleotide sequences which are sufficiently complementary to form complexes via Watson-Crick base pairing. Where a primer (or splice template) “hybridizes” with target (template), such complexes (or hybrids) are sufficiently stable to serve the priming function required by the DNA polymerase to initiate DNA synthesis.

As used herein, the term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term “gene” encompasses both cDNA and genomic forms of a gene. A genomic form or clone of a gene contains the coding region interrupted with non-coding sequences termed “intervening regions” or “intervening sequences.”

As used herein, the term “gene silencing” refers to a phenomenon whereby a function of a gene is completely or partially inhibited. Throughout the specification, the terms “silencing,” “inhibition,” “quelling,” “knockout” and “suppression,” when used with reference to gene expression or function, are used interchangeably.

As used herein, the term “oligonucleotide” is defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

As used herein, the term “transfection” refers to the introduction of foreign DNA into eukaryotic cells. Transfection can be accomplished by a variety of means known to the art, including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

A primer is selected to be “substantially” or “sufficiently” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

As used herein, the term “amplification” refers to nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that will process only specific sequences of nucleic acid in a heterogeneous mixture of nucleic acid. For example, in the case of Qb replicase, MDV-1 RNA is the specific template for the replicase (Kacian et al. (1972) Proc. Natl. Acad. Sci. USA 69, 3038). Other nucleic acid will not be replicated by this amplification enzyme. Similarly, in the case of T7 RNA polymerase, this amplification enzyme has a stringent specificity for its own promoters (Chamberlin et al. (1970) Nature 228, 227). Taq and Pfu polymerases, by virtue of their ability to function at high temperature display high specificity for the sequences bounded, and thus defined by the primers.

As used herein, the terms “amplifiable nucleic acid” and “amplified products” refer to nucleic acids which may be amplified by any amplification method.

As used herein, the term “probe” refers to an oligonucleotide (i.e., a sequence of nucleotides) which is capable of hybridizing to another oligonucleotide of interest, whether occurring naturally as in a purified restriction digest or produced synthetically, recombinantly or by enzymatic amplification. A probe may be single-stranded or double-stranded. Probes are useful in the detection, identification and isolation of particular gene sequences.

As used herein, the term “enzymatic amplification” (such as PCR, NASBA and RNA-PCR) refers to a method for increasing the concentration of a segment in a target sequence from a mixture of genomic DNAs without cloning or purification (U.S. Pat. Nos. 4,683,195; 4,683,202; 4,965,188 (PCR); 5,888,779 (NASBA); 6,197,554 (RNA-PCR) and WO 00/75356, hereby incorporated by reference). This process for amplifying the target sequence consists of introducing a large excess of two oligonucleotide primers to the DNA mixture containing the desired target sequence, followed by a precise sequence of thermal cycling in the presence of DNA and/or RNA polymerase(s). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. Because the desired amplified segments of the target sequence become the predominant sequences (in terms of concentration) in the mixture, they are said to be amplified.

With enzymatic amplification, it is possible to amplify a single copy of a specific target sequence in genomic DNA to a level detectable by several different methodologies (e.g., incorporation of biotinylated primers followed by avidin-enzyme conjugate detection; incorporation of 32 P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR and RNA-PCR process itself are, themselves, efficient templates for subsequent PCR and RNA-PCR amplifications.

As used herein, the term “portion” when in reference to a protein or nucleic acid sequence refers to fragments of that protein or nucleic acid sequence. Fragments of a protein can range in size from four amino acid residues to the entire amino acid sequence minus one amino acid.

The term “nucleotide analog” as used herein refers to modified or non-naturally occurring nucleotides such as deoxyuracil, inosine, xanthine, hypoxanthine, labeled nucleotides, 7-deaza-dNTP, methylthio-linked nucleotide, phosphothio-linked nucleotide, hexose-containing nucleotide, morpholino nucleotide, peptide nucleic acid (PNA), etc. Nucleotide analogs include base analogs and comprise modified backbone forms of deoxyribonucleotides as well as ribonucleotides, such as ribonucleotide(s) in a DNA sequence and deoxyribonucleotide(s) in an RNA sequence.

The term “Northern blot,” as used herein refers to the analysis of RNA by electrophoresis of RNA on agarose gels to fractionate the RNA according to size, followed by transfer of the RNA from the gel to a solid support such as nitrocellulose or a nylon membrane. The immobilized RNA is then probed with a labeled probe to detect RNA species complementary to the probe used. Northern blots are a standard tool of molecular biologists (Sambrook et al., (1989) Molecular Cloning, 2^{nd} Ed., Cold Spring Harbor Laboratory Press, pp 7.39-7.52).

As used herein, the term “Southern blot” refers to the analysis of DNA on agarose or acrylamide gels to fractionate the DNA according to size, followed by transfer of the DNA from the gel to a solid support such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., supra).

The term “virus” refers to obligate, ultramicroscopic, intracellular parasites incapable of autonomous replication (i.e., replication requires the use of the host cell's machinery).

As used herein, the terms “Taq-like polymerase” and “Taq polymerase” refer to Taq DNA polymerase and derivatives. Taq DNA is widely used in molecular biology techniques including recombinant DNA methods. For example, various forms of Taq have been used in a combination method which utilizes PCR and reverse transcription (See e.g., U.S. Pat. No. 5,322,770, incorporated herein in its entirety by reference). DNA sequencing methods which utilize Taq DNA polymerase have also been described. (See e.g., U.S. Pat. No. 5,075,216, incorporated herein in its entirety by reference).

As used herein, the terms “Tth-like polymerase” and “Tth polymerase” refer to polymerase isolated from Thermus thermophilus. Tth polymerase is a thermostable polymerase that can function as both reverse transcriptase and DNA polymerase (Myers and Gelfand, (1991) Biochemistry 30, 7662-7666). It is not intended that the methods of the present invention be limited to the use of Taq-like or Tth-like polymerases. Other thermostable DNA polymerases which have 5′ to 3′ exonuclease activity (e.g., Tma, Tsps17, TZ05, Tth and Taf) can also be used to practice the compositions and methods of the present invention.

As used herein, “reverse transcription” means the synthesis of a DNA molecule from an RNA molecule using an enzymatic reaction in vitro. For example, the RNA molecule may be primed with a primer that is complementary to the RNA molecule and the DNA molecule is synthesized by extending using a reverse transcriptase such as Tth DNA polymerase with reverse transcription activity, MMLV reverse transcriptase, AMV reverse transcription, and any other enzymes that have the ability to synthesize DNA molecule from an RNA molecule as template.

As used herein, “in vitro transcription” means the synthesis of an RNA molecule from a nucleic acid template molecule using an enzymatic reaction in vitro. For example, the nucleic acid template may be a double-stranded DNA sequence and comprises an RNA polymerase promoter such as T7, SP6, T3, or any other enzyme promoter for synthesis of RNA from the template.

### EXAMPLES

The following examples serve to illustrate certain preferred embodiments and aspects of the present invention and are not to be construed as limiting the scope thereof.

In the experimental disclosure which follows, the following abbreviations apply: M (molar); mM (millimolar); mm (micromolar); mol (moles); pmol (picomolar); gm (grams); mg (milligrams); L (liters); ml (milliliters); ml (microliters); ° C. (degrees Centigrade); cDNA (copy or complementary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); NaCl (sodium chloride); HEPES (N-2-hydroxyethylpiperazine-N-2-ethanesulfonic acid); HBS (HEPES buffered saline); SDS (sodium dodecylsulfate); Tris-HCl (tris-hydroxymethylaminomethane-hydrochloride); and ATCC (American Type Culture Collection, Rockville, Md.).

All routine techniques and DNA manipulations, such as gel electrophoresis, were performed according to standard procedures. (See Sambrook et al., supra). All enzymes and buffer treatments were applied following the manufacture's recommendations (Roche Biochemicals, Indianapolis, Ind.). For Northern blots, mRNAs were fractionated on 1% formaldehyde-agarose gels and transferred onto nylon membranes (Schleicher & Schuell, Keene, N.H.). Probes were labeled with the Prime-It II kit (Stratagene, La Jolla, Calif.) by random primer extension in the presence of [³²P]-dATP (>3000 Ci/mM, Amersham International, Arlington Heights, Ill.), and purified with Micro Bio-Spin chromatography columns (BIO-RAD, Hercules, Calif.). Hybridization was carried out in the mixture of 50% freshly deionized formamide (pH 7.0), 5× Denhardt's solution, 0.5% SDS, 4×SSPE and 250 mg/ml denatured salmon sperm DNAs (18 hr, 42° C.). Membranes were sequentially washed twice in 2×SSC, 0.1% SDS (15 min, 25° C.), and once each in 0.2×SSC, 0.1% SDS (15 min, 25° C.); and 0.2×SSC, 0.1% SDS (30 min, 65° C.) before autoradiography by films.

For cell fixation and permeabilization, MCF-7 cells, a breast cancer cell line, were grown in MEM medium supplemented with 10% fetal calf serum. A sample containing cells cultured in a 60 mm dish (70% full of cells) was trypsinized, collected and washed three times in 5 ml phosphate buffered saline (PBS, pH 7.2) at room temperature. After washing, the cells were suspended in 1 ml of ice-cold 10% formaldehyde solution in 0.15M NaCl. After one hour incubation on ice with occasional agitation, the cells were centrifuged at 13,000 rpm for 2 min, and washed three times in ice-cold PBS with vigorous pipetting. The collected cells were resuspended in 0.5% non-ionic detergents, such as (octylphenoxy)-polyethanol or polyoyethylenesorbitan (Sigma), and incubated for one hour with frequent agitation. The cells were washed three times in ice-cold PBS containing 0.1 M glycine, then resuspended in 1 ml of the same buffer with vigorous pipetting in order to be evenly separated into small aliquots and stored at −70° C. for up to a month.

### Example 1

### Cell Fixation and Permeabilization

LNCaP cells, a prostate cancer cell line, were grown in RPMI 1640 medium supplemented with 2% fetal calf serum. A sample containing cells cultured in a 60 mm dish (70% full of cells) was trypsinized, collected and washed three times in 5 ml phosphate buffered saline (PBS, pH 7.2) at room temperature. After washing, the cells were suspended in 1 ml of ice-cold 10% formaldehyde solution in 0.15M NaCl. After one hour incubation on ice with occasional agitation, the cells were centrifuged at 13,000 rpm for 2 min, and washed three times in ice-cold PBS with vigorous pipetting. The collected cells were resuspended in 0.5% nonionic detergents, such as (octylphenoxy)-polyethanol or polyoyethylenesorbitan (Sigma), and incubated for one hour with frequent agitation. The cells were washed three times in ice-cold PBS containing 0.1M glycine, then resuspended in 1 ml of the same buffer with vigorous pipetting in order to be evenly separated into small aliquots and stored at −70° C. for up to a month.

### Example 2

### In-Cell Reverse Transcription and Poly-(N) Tailing of cDNAs

For reverse transcription of mRNAs in cells, twenty of the fixed cells were thawed, resuspended in 20 μl of ddH₂O, heated to 65° C. for 3 min and then cooled on ice. A 50 μl RT reaction was prepared, comprising 5[α]of 10× in-cell RT buffer (1.2M KCl, 0.5M Tris-HCl, 80 mM MgCl₂, 10 mM dithiothreitol, pH 8.1 at 42° C.), 5 μl of 5 mM dNTPs, 25 pmol oligo(dT)n-T7 promoter (SEQ ID NO. 1), 80U RNase inhibitor and above cold cells. After reverse transcriptase (40U) was added, the RT reaction was mixed and incubated at 55° C. for three hours. The cells were then washed once with PBS and resuspended in a 50 μl tailing reaction, comprising 2 mM dGTP, 10 μl of 5× tailing buffer (250 mM KCl, 50 mM Tris-HCl, 7.5 mM MgCl₂, pH 8.3 at 20° C.). The tailing reaction was heated at 94° C. for 3 min and then chilled in ice for mixing with terminal transferase (20U), following further incubation at 37° C. for 20 min. Final reaction was stopped at 94° C. for 3 min. The reaction mixture was chilled in ice immediately, which formed the poly(N)-tailed cDNAs.

### Example 3

### Single-Cell mRNA Amplification

To increase the intracellular copies of whole mRNAs, the T7 promoter region of a poly(N)-tailed cDNA was served as a coding strand for the amplification by T7 RNA polymerase (Eberwine et al., Proc. Natl. Acad. Sci. USA 89: 3010-3014 (1992)). As few as one cell in 5 μl of above tailing reaction can be used to accomplish full-length aRNA amplification. An in-cell transcription reaction was prepared on ice, containing 25 pmol poly(dC)-12mer primer (SEQ ID NO. 2), 1 mM dNTPs, Pwo DNA polymerase (5U), 5 μl of 10× Transcription buffer (Roche), 2 mM rNTPs and T7 RNA polymerase (2000U). The hybridization of 20mer primer to the poly(N)-tailed cDNAs was incubated at 65° C. for 5 min to complete second strand cDNA synthesis and then RNA polymerase was added to start transcription. After four hour incubation at 37° C., the cDNA transcripts were isolated from both cells and supernatant, to be directly used in the following reverse transcription. The reaction was finally stopped at 94° C. for 3 min and chilled in ice.

### Example 4

### In Vitro Reverse Transcription and PCR Amplification

A 50 μl RT reaction was prepared, comprising 5 μl of 10×RT buffer (300 mM KCl, 0.5M Tris-HCl, 80 mM MgCl₂, 10 mM dithiothreitol, pH 8.3 at 20° C.), 5 μl of 5 mM dNTPs, 25 pmol oligo(dC)₁₀-T7 promoter mix (SEQ ID NO. 3, 4 and 5), 80U RNase inhibitor, ddH₂O and 5 μl of the above aRNA containing supernatant. After reverse transcriptase (40U) was added, the RT reaction was vortexed and incubated at 55° C. for three hours. The resulting products of RT can be directly used in following PCR reaction (50 μl), comprising 5±1 of 10×PCR buffer (Roche), 5 μl of 2 mM dNTPs, 25 pmol T720mer primer, 25 pmol poly(dT)-26mer primer (SEQ ID NO. 6), ddH₂O, 5 μl of above RT product and 3U of Taq/Pwo long-extension DNA polymerase. The PCR reaction was subjected to thirty cycles of denaturation at 95° C. for 1 min, annealing at 55° C. for 1 min and extension at 72° C. for 3 min. The quality of final amplified cDNA library (20 μl) was assessed on a 1% formaldehyde-agarose gel, ranging from 100 bp to above 12 kb.

### Example 5

### RNA-PCR

Pre-cycling procedures. Primers used in RNA-PCR were as follows: a poly(dT)-26 primer (5′-TTTTTTTTTT TTTTTTTTTT TTTTTT-3′) (SEQ ID NO. 6) and an oligo(dC)₁₀N-promoter primer mixture comprising equal amounts of oligo(dC)₁₀-GT7 primer (5′-dCCAGTGAATT GTAATACGAC TCACTATAGG GAAC₁₀G-3′) (SEQ ID NO. 3); oligo(dC)₁₀ A-T7 primer (5′-dCCAGTGAATT GTAATACGAC TCACTATAGG GAAC₁₀A-3′) (SEQ ID NO. 4); and oligo(dC)₁₀T-T7 primer (5′-dCCAGTGAATT GTAATACGAC TCACTATAGG GAAC₁₀T-3′) (SEQ ID NO. 5). The poly(dT)-26 primer was used to reverse transcribe mRNAs into first-strand cDNAs, while the oligo(dC)₁₀N-promoter primers functioned as a forward primer for second-strand cDNA extension from the poly(dG) end of the first-strand cDNAs and therefore RNA promoter incorporation. All oligonucleotides were synthetic and purified by high performance liquid chromatography (HPLC).

For in situ hybridization and cell preparations, fresh formaldehyde prefixed paraffin-embedded sections were dewaxed, dehydrated and refixed with 4% PFA, and then permeabilized with proteinase K (10 μg/ml; Roche) after rinsing with 1×PBS. In situ hybridization was achieved with a denatured hybridization mixture within a 200 μl coverslip chamber, containing 40% formamide, 5×SSC, 1× Denhardt's reagent, 50 μg/ml salmon testis DNA, 100 μg/ml tRNA, 120 pmol/ml poly(dT)-26 primer, 10 pmol/ml biotin-labeled activin antisense probe (˜700 bases in size) and tissue. After 10 h incubation at 65° C., sections were washed once with 5×SSC at 25° C. for 1 h and once with 0.5×SSC, 20% formamide at 60° C. for 30 min to remove unbound probes. A pre-heating step (68° C., 3 min) immersing the sections in a mild denaturing solution (25 mM Tris-HCl, pH 7.0, 1 mM EDTA, 20% formamide, 5% DMSO and 2 mM ascorbic acid) was performed to minimize secondary structures (including crosslinks) and to reduce the background. After the temperature was lowered to 45° C., 2,5-diaziridinyl-1,4-benzoquinone (200 μM; Sigma Chemical Co., St. Louis, Mo.) was added to each incubation for a further 30 min. Finally, 0.1×SSC, 20% formamide was applied at 60° C. for 30 min to clean sections for chromogenic detection with straptavidin-alkaline phosphatase and Fast Red staining (Roche Biochemicals, Indianapolis, Ind.). Positive and negative results were observed and recorded under a microscope. RNase-free enzymes and DEPC-treated materials were required throughout the procedure.

RNA-PCR. For amplification of intracellular mRNAs, more than 20 fixed cells were preheated at 94° C. for 5 min and applied to a reverse transcription (RT) reaction mixture (50 μl) on ice, comprising 10 μl of 5×RT&T buffer [100 mM Tris-HCl, pH 8.5 at 25° C., 600 mM KCl, 300 mM (NH₄)₂SO₄, 25 mM MgCl₂, 5 M betaine, 35 mM dithiothreitol, 10 mM spermidine and 25% dimethylsulphoxide (DMSO)], 1 μM poly(dT)-26 primer, dNTPs (1 mM each dATP, dGTP, dCTP and dTTP) and RNase inhibitors (10 U). After 6 U Caxboxydothernius hydrogenoformans (C. therm.) polymerase (Roche) was added, the reaction was incubated at 52° C. for 3 min and shifted to 65° C. for another 30 min. The first-strand cDNAs so obtained were collected with a Microcon-50 microconcentrater filter, washed once with 1×PBS and suspended in a tailing reaction (50 μl), comprising 10 μl of 5× tailing buffer (250 mM KCl, 100 mM Tris-HCl, 4 mM CoCl₂, 10 mM MgCl₂, pH 8.3 at 20° C.) and 0.5 mM dGTP. After 75 U terminal transferase (Roche) was added, the reaction was incubated at 31° C. for 15 min, stopped by denaturation at 94° C. for 2 min and instantly mixed with 1 μM oligo(dC)₁₀-T7 primer mixture. After briefly centrifuging, 3.5 U Taq DNA polymerase (Roche) and 1 mM of each of the dNTPs was added to form promoter-linked double-stranded cDNAs at 52° C. for 3 min, and then 72° C. for 7 min. The cells were broken by adding 1 vol of 2% (octylphenoxy)-polyethanol polyoyethylenesorbitan for 10 min, and then the double-stranded cDNAs were washed and recollected with a microcon-50 in autoclaved ddH₂O. This completed the pre-cycling steps for the following cycling amplification.

A transcription reaction (50 μl) was prepared, containing 10 μl of 5×RT&T buffer, rNTPs (1 mM each ATP, GTP, CTP and UTP), RNA inhibitors (10 U), T7 RNA polymerase (200 U; Roche) and the double-stranded cDNAs. After 2 h incubation at 37° C., the cDNA transcripts were isolated with a microcon-50 filter in 20 μl of DEPC-treated TE buffer (pH 7.0) and used directly for the next round of RNA-PCR without the tailing reaction, containing 10 μl of 5×RT&T buffer, 1 μM poly(dT)-26 primer, 1 μM oligo(dC)₁₀-T7 primers, dNTPs (1 mM each), rNTPs (1 mM each), C. therm. polymerase, Taq DNA polymerase and the transcription products (20 pg). T7 RNA polymerase was renewed in every transcription step due to prior denaturation. The quality of mRNA products (20 μg) after three rounds of amplification was assessed on a 1% form aldehyde-agarose gel. products (20 μg) after three rounds of amplification was assessed on a 1% form aldehyde-agarose gel.

### Example 6

### Thermostable Cycling Amplification Procedure

Few fixed and permeabilized cells were applied to a reaction mixture (20 ml) on ice, comprising 2 ml of 10×RT&T buffer (400 mM Tris-HCl, pH 8.3 at 25° C., 400 mM NaCl, 80 mM MgCl₂, 5M betaine, 100 mM DTT and 20 mM spermidine), 1 mM Shh-antisense primer (SEQ ID. NO. 7), 1 mM Shh-sense promoter-primer (SEQ ID. NO. 8), 2 mM rNTPs, 2 mM dNTPs and RNase inhibitors (10U). After C. therm./Taq DNA polymerase mixture (4U) was added, the reaction was incubated at 52° C. for 3 min, at 65° C. for 30 min, at 94° C. for 3 min, at 52° C. for 3 min, and then at 68° C. for 3 min. A transcription reaction was prepared by adding T7 RNA polymerase (200U) and C. therm. polymerase (6U) mixture into above reaction. After one hour incubation at 37° C., the resulting mRNA transcripts were continuously reverse-transcribed into mRNA-cDNA duplexes at 52° C. for 3 min, and then at 65° C. for 30 min, so as to provide sRNA-cDNA hybrids. The quality of amplified mRNA-cDNA products can be assessed on a 1% formaldehyde-agarose gel (Lin et al., Nucleic Acid Res. (1999)).

### Example 7

### Liposomal Transfection Procedure

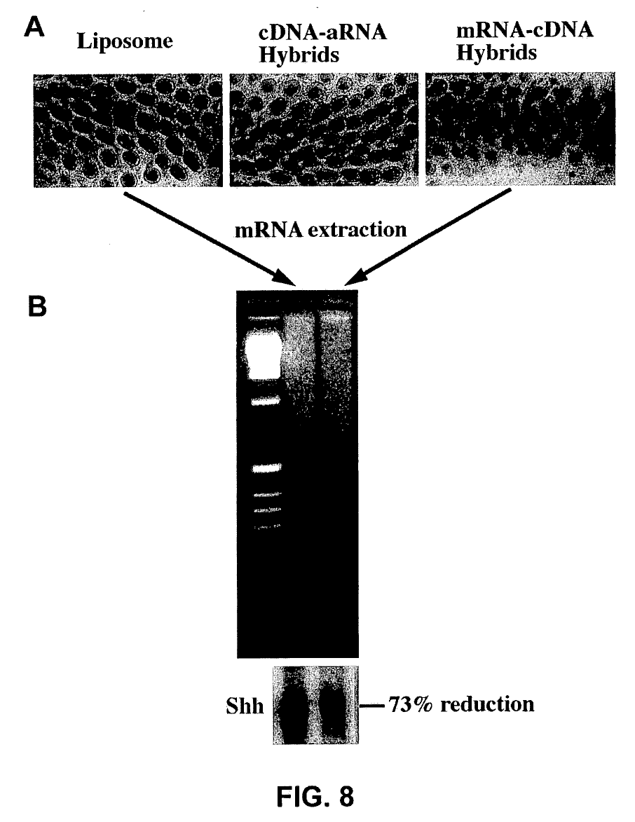

An sRNA-aDNA hybrid Shh probe (10 mg) was dissolved in 75 ml of Hepes buffer (pH 7.4). The resulting solution was mixed with 50 ml of DOTAP® liposome (1 mg/ml, Roche Biochemicals) on ice for 30 min., then subsequently applied to 60 mm diameter culture dishes containing four or five chicken skin explants. The skin explants were grown in HBSS medium. After a 36 hour incubation, the disturbance of feather growth was observed only in the sRNA-aDNA hybrid set while the blank-liposomal control have no effects (FIG. 8A). The Northern blot results of blank control and sRNA-aDNA (as shown in mRNA-cDNA) hybrid set showed that a 73% gene silencing effect occurred by treating the sRNA-aDNA hybrid Shh probes (FIG. 8B).

### Example 8

### Gene Silencing Using a Chicken Embryo Model

This Example shows the effectiveness of a sRNA-aDNA strategy to knockout gene expression in vivo, using a developing chicken embryo as a model. In this example, β-catenin expression was targeted in the skin and liver of developing chick embryos. The sRNA-aDNA duplexes used for knocking β-catenin expression in vivo can be generated using the improved RNA-PCR technology discussed above.

For β-catenin, a double-stranded DNA template fragment, a pair of primers was designed based on the cDNA sequence. The central region for knockout targeting of β-catenin (aa 306-644) required four primers (i.e., primers A-D). The upstream (A) primer comprises the sequence 5′-ATGGCAATCAAGAAAGTAAGC-3′ (SEQ ID. NO. 9). The downstream (B) primer comprises the sequence 5′-GTACAACAACTGCACAAATAG-3′ (SEQ ID. NO. 10). Another set of primers was required for the generation of the desired duplexes. The (C) primer was generated by adding the T7 promoter (RP) before the 5′ end of the (A) primer. The (D) primer was generated by adding the T7 promoter before the 5′ end of the (B) primer.

For sRNA-aDNA templates, B and C primers were used as primers in a polymerase chain reaction to generate promoter-linked double stranded cDNA. The promoter-linked double stranded cDNA was transcribed with T7 RNA polymerase for 2 h, and AMV reverse transcriptase for 1 hour. Subsequently, the D-RNAi hybrids were collected by filtration over a Microcon 50 (Amicon, Bedford, Mass.) column and eluted with 20 μl of elution buffer (20 mM HEPES). The final concentration of D-RNAi is approximately 25 nM.

For the sDNA-aRNA template, A and D primers were used in a similar procedure as described above in the opposite orientation. The size of the hybrids was then determined on a 1% agarose gel. The hybrids were kept at −20° C. until use.

Fertilized eggs were obtained from SPAFAS farm (Preston, Conn.) and incubated in humidified incubator (Humidaire, New Madison, Ohio). At designated dates, eggs were put under a dissection microscope and the egg shells were sterilized. The shells were carefully cracked open and a window was made to get access to the embryos.

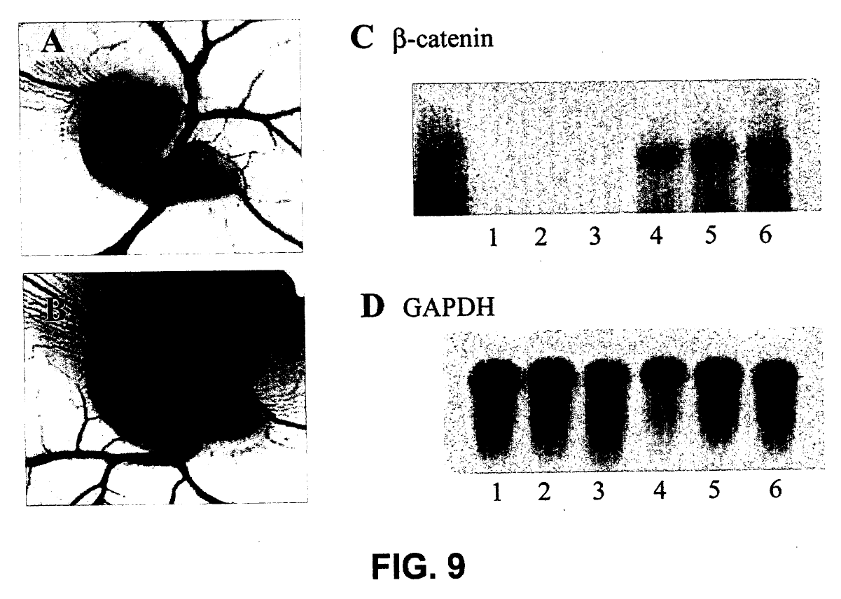

Using embryonic day three chicken embryos, either sRNA-aDNA or sDNA-aRNA (25 nM) was injected into the ventral body cavity, close to where the liver primordia would form. The sRNA-aDNA hybrid was mixed with DOTAP liposome (Roche, Indianapolis, Ind.) at a ratio of 3:2. A 10% (v/v) fast green solution was added before the injection to increase visibility (FIG. 9B). The mixtures were injected into the ventral side near the liver primordia and below the heart using heat pulled capillary needles. After injection, the eggs were sealed with scotch tape and put back into a humidified incubator (Lyon Electric Company, Chula Vista, Calif.) at 39-40° C. until the harvesting time.

At designated days after injection, the embryos were removed, examined and photographed under a dissection microscope. While there are malformations, the embryos survived and there was no overt toxicity or overall perturbation of embryo development. The liver was closest to the injection site and is most dramatically affected in its phenotypes. Other regions, particularly the skin, are also affected by the diffused nucleotides.

Selected organs were removed and total RNAs were collected with an RNeasy kit (Qiagen, Valencia, Calif.) for Northern analysis. RNAs were fractionated in an RNase free polyacrylamide gel (1%) and then transferred to Nylon membranes for 16-18 h. The tested gene was hybridized with a radiolabeled probe, and an autoradiograph was exposed. Northern blot hybridizations using RNA from dissected livers showed that β-catenin in the control livers remained expressed (lane 4-6, FIG. 9C), whereas the level of 13-catenin mRNA was decreased dramatically (lane 1-3, FIG. 9D) after treatment with D-RNAi directed against β-catenin. In this figure, C is hybridized to a β-catenin probe, while D is hybridized to a GAPDH probe, to show that equivalent concentrations were loaded. Controls used include liposome alone and similar concentrations of perfectly matched sDNA-aRNA.

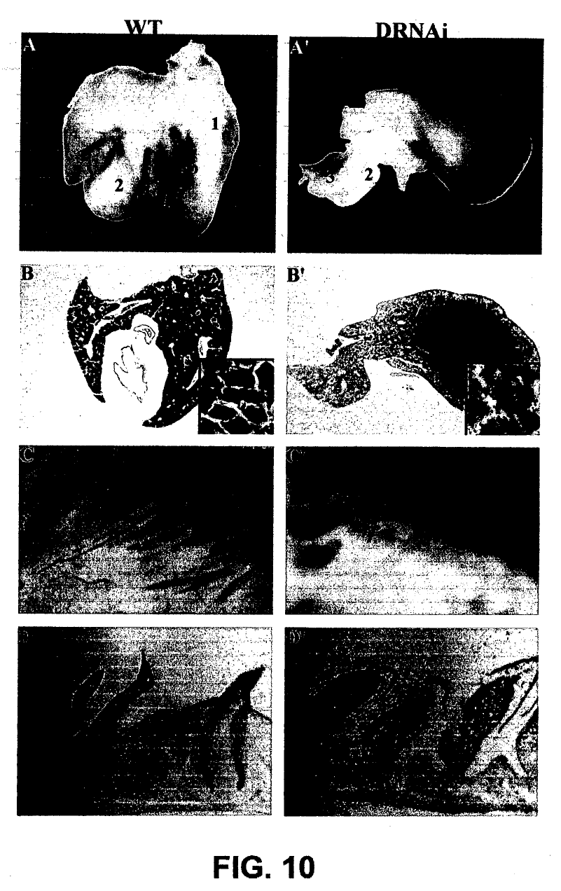

Livers after ten days of injection with sRNA-aDNA duplex showed an enlarged and engorged first lobe, but the size of the second and third lobes of the livers were dramatically decreased (FIGS. 10A-A′). Histological sections of normal liver showed hepatic cords and sinusoidal space with few blood cells. In the β-catenin treated embryos, the general architecture of the hepatic cells in lobes 2 and 3 remained unchanged. However, in lobe 1 there are islands of abnormal regions. The endothelium development appears to be defective and blood is outside of the blood vessels. Abnormal types of hematopoietic cells are observed between the space of hepatocytes, particularly dominated by a population of small cells with round nuclei and scanty cytoplasm. In severely affected areas, hepatocytes were disrupted (FIGS. 10B, B′).

Since skin is exposed in the amniotic cavity and is most accessible to the nucleotides that leaked out, patches of skin that showed phenotypes were also observed. At embryonic day 13, skin should have formed elongated feather buds, with a primordial blood vessel running into its mesenchymal core. In the sRNA-aDNA β-catenin affected region, feather buds become engorged with blood, starting from the distal end of the feather tip (FIGS. 10C, C′). The adjacent skin was normal (not shown), and works as a good control. Histological sections showed that the normal feather buds have continued their morphogenetic process with the epidermis invaginated to form the feather follicle walls, surrounding a mesenchymal core. In affected areas, the distal feather bud mesenchyme was full of engorged blood vessels and blood cells. Distal epidermis also detached from the feather mesenchyme, and proximal epidermis failed to invaginate to form follicles (FIGS. 10D, D′).

### Example 9

### Generation of bcl-2 sRNA-aDNA Hybrids

Four synthetic oligonucleotides were used in the generation of bcl-2 sRNA-aDNA hybrids as follows: T7-bcl-2 primer (5′-dAAACGACGGC CAGTGAATTG TAATACGACT CACTATAGGC GGATGACTGA GTACCTGAAC CGGC-3′) (SEQ ID. NO. 11) and anti-bcl2 primer (5′-dCTTCTTCAGGCCAGGGAGGCATGG-3′) (SEQ ID. NO. 12) for mRNA-cDNA hybrid (D-RNAi) probe preparation; T7-anti-bcl2 primer (5′-dAAACGACGGC CAGTGAATTG TAATACGACT CACTATAGGC CTTCTTCAGG CCAGGGAGGC ATGG-3′) (SEQ ID NO. 13) and bcl2 primer (5′-dGGATGACTGAGTACCTGAACCGGC-3′) (SEQ ID NO. 14) for antisense RNA (aRNA)-cDNA hybrid (reverse D-RNAi) probe preparation. The design of the sequence-specific primers is based on the same principle used by PCR (50˜60% G-C rich), while that of the promoter-linked primers however requires a higher G-C content (60˜65%) working at the same annealing temperature as above sequence-specific primers due to their unmatched promoter regions. For example, new annealing temperature for the sequence-matched region of a promoter-linked primer is equal to [2° C.×(dA+dT)+3° C.×(dC+dG)]×5/6, not including the promoter region. All primers were purified by polyacrylamide gel electrophoresis (PAGE) before use in RNA-PCR reaction.

### Example 10

### Treatment of LNCaP Cells to Induce bcl-2 Expression

LNCaP cells were obtained from the American Type Culture Collection (ATCC, Rockville, Md., and grown in RPMI 1640 medium supplemented with 10% fetal bovine serum with 100 μg/ml gentamycin at 37° C. under 10% CO₂. These cultured cells were treated with one dose of 100 nM 5α-anrostan-17β-ol-3-one to induce bcl-2 expression. For liposomal transfection of anti-bcl-2 probes, the probes (5 nM) in DOTAP liposome (Roche Biochemicals) were applied to a 60 mm culture dish which contained LNCaP cells at 15% confluency. After a 18-hour incubation, the cells took up about 60% of the probe-containing liposome. Uptake improved to 100% after 36 hours of incubation. The addition of α-amanitin was completed at the same time as the liposomal transfection. The apoptotic effect of phorbol-12-myristate-13-acetate (10 mM) was initiated at 12 hours after liposomal transfection. The mRNAs from the transfected LNCaP cells were isolated by poly(dT) dextran columns (Qiagen, Santa Clarita, Calif.), fractionated on a 1% formaldehyde-agarose gel after a 36 hour incubation period, and transferred onto nylon membranes. After 48-hour transfection, genomic DNAs were isolated by an apoptotic DNA ladder kit (Roche Biochemicals) and assessed on a 2% agarose gel. Cell growth and morphology were examined by microscopy and cell counting, following known techniques. (See e.g., Lin et al., Biochem. Biophys. Res. Commun. 281: 639-644 (2001) & Current Cancer Drug Targets 1: 241-247 (2001)).

### Example 11

### Probe Preparations from Androgen-Treated LNCaP Cells

For the generation of RNA-DNA hybrid probes, an RNA-polymerase cycling reaction (RNA-PCR) procedure was modified to generate either sRNA-aDNA or cDNA-aRNA hybrids. Total RNAs (0.2 μg) from androgen-treated LNCaP cells were applied to a reaction (50 μl in total) on ice, comprising 5 μl of 10×RT&T buffer (400 mM Tris-HCl, pH 8.3 at 25° C., 400 mM NaCl, 80 mM MgCl₂, 2 M betaine, 100 mM DTT and 20 mM spermidine), 1 M sequence-specific primer for reverse transcription, 1 μM promoter-linked primer for cDNA-doublestranding, 2 mM rNTPs, 2 mM dNTPs and RNase inhibitors (10 U). After C. therm./Taq DNA polymerase mixture (4 U each) was added, the reaction was incubated at 52° C. for 3 min, 650° C. for 30 min, 940° C. for 3 min, 520° C. for 3 min and then 680° C. for 3 min. This formed a promoter-linked double-stranded cDNA for next step of transcriptional amplification up to 2000 fold/cycle. An in-vitro transcription reaction was performed by adding T7 RNA polymerase (160 U) and C. therm. polymerase (6 U) into above reaction. After one hour incubation at 37° C., the resulting mRNA transcripts were continuously reverse-transcribed into mRNA-cDNA duplexes at 52° C. for 3 min and then 650° C. for 30 min, so as to form sRNA-aDNA hybrids. The generation of sDNA-aRNA hybrids was the same procedure as aforementioned except using 1 μM sequence-specific primer for cDNA-double-stranding and 1 μM promoter-linked primer for reverse transcription. The RNA-PCR procedure can be reiterated to produce enough RNA-DNA hybrids for gene silencing analysis. For the preparation of double-stranded RNA probes, complementary RNA products were transcribed from both orientations of above promoter-linked double-stranded cDNAs and mixed together without reiterating reverse transcription activity. The quality of amplified probes were assessed on a 1% formaldehyde-agarose gel.

### Example 12

### Gene Silencing Using DNA-RNA Hybrids: In Vitro Breast Cancer Model

As noted earlier, posttranscriptional gene silencing (PTGS) and RNA interference (RNAi) have been found capable of quelling specific gene activities in a variety of in vivo systems.

According to the invention provided herein, ectopic transfection of a sequence-specific DNA-RNA hybrid (instead of a transgene dsDNA or dsRNA) is used to induce intracellular gene silencing in human cells. Although previous transgene/dsRNA transfection experiments showed that PTGS/RNAi effects are limited to plants and some simple animals, using the present invention, specific gene interference of β-catenin expression in human MCF-7 breast cancer cells using the cDNA-aRNA hybrid transfection has been successfully detected.

Normal human mammary granular cells do not express β-catenin protein, whereas neoplastic breast tissues from late-stage patients show a highly elevated level of this proliferation-stimulating oncoprotein. The malignancy and metastatic potentials of the breast cancer cells are also significantly increased after β-catenin expression. It is known in the art that over-expression of β-catenin protects malignant cancer cells from apoptosis and confers resistance to many anti-cancer drugs in vivo. To overcome such resistance, transcriptional knock down or knockout gene therapy may provide a counteract control for the expression of β-catenin.

The potential utility of DNA-RNA transfection in preventing oncogene expression was therefore tested on β-catenin-expressing MCF-7 cells, expecting to reduce β-catenin protein amount and increase cancer cell susceptibility to apoptotic stimuli. Following our previous findings, MCF-7 cells were treated with different dosages of anti-β-catenin sense DNA-antisense RNA (sDNA-aRNA) hybrids (5 nM at an optimally effective concentration and 50 nM at a ten-fold high concentration). FIG. 12(a) shows the immunostaining results of expressed β-catenin protein in red ACE substrate color. At 5 nM concentration of the sDNA-aRNA transfection (n=4), the expression rate was decreased from 38.8±3.1% (control) to 13.3±2.8% (transfected) cell population, indicating a 65.8% reduction. At 50 nM concentration (n=5), the expression rate was decreased from 53.5±3.6% (control) to only 16.5±3.1% (transfected) cell population, indicating a 69.3% reduction.

The silencing of β-catenin expression also decrease the proliferation rate of cancer cells. At 5 nM concentration of the sDNA-aRNA transfection (n=4), the density of cell population was decreased from average 112 (control) to 43 cell/mm³ (transfected), indicating a 62.7% reduction. At 50 nM concentration (n=5), the density of cell population was decreased from average 155 (control) to only 37 cell/mm³ (transfected) cell population, indicating a 76.2% reduction. It is also noted that the cell morphology of all four sets is the same, without the debris of apoptotic bodies (interferon-caused cell death). Such findings suggest that the sDNA-aRNA transfection can successfully knock out average 67% of β-catenin oncogene expression and inhibit more than 62% cancer cell growth without the induction of cytotoxicity. Contrary to previous dsRNA reports, dsRNA transfection usually causes a very significant interferon-induced cytotoxicity at the concentrations more than 10 nM.

The increase of RNA-directed endoribonuclease (RDE) activity is also detected after sDNA-aRNA transfections. As noted earlier, the RDE is required for the onset of PTGS/RNAi phenomena in many in cell and in vivo systems. The activity of RDE is measured by adding 2 μl cell extracts into 21 g of 1 kb dsRNA preparations for 10 min at 25° C. Since the dsRNA is labeled by [³³P]-CTP (>3000 Ci/mM, Amersham International), the degradation rate can be easily observed by 1% agarose gel electrophoresis, blot transferring and then film exposure. The bar chart of FIG. 12(b) shows the RDE activity in black bars and the gene silencing rate in white bars. At 5 nM concentration of the sDNA-aRNA transfection (n=4), the RDE activity was promoted from 54.2 (control) to 90.6 ng/min (transfected), indicating a 167% increase rate. At 50 nM concentration (n=5), the RDE activity was promoted from 53.2 (control) to 92.7 ng/min (transfected), indicating a 174% increase rate. This data suggests that the sDNA-aRNA transfection induce a gene-specific silencing effect through the PTGS/RNAi phenomena.

There are three major effects of PTGS, i.e., initiation, spreading and maintenance, all of which are also found in many inheritable RNAi phenomena. The initiation indicates that the onset of PTGS/RNAi takes a relatively long period of time (1˜3 days) to develop enough small RNA or short interfering RNA (si-RNA) for specific gene knockout. With traditional antisense transfection processes, it only takes several hours to reach the same gene silencing results but with much higher dosages and higher cytotoxicity. Also, unlike the short-term effectiveness of traditional antisense transfections, the PTGS/RNAi effects may spread from a transfected cell to neighboring cells and can be maintained for a very long time (weeks to lifetime) in a mother cell as well as its daughter cells (Grant (1999) supra). Based on these features, a more efficient and reliable gene therapy is expected.

### The Preparation of sDNA-aRNA Hybrids for β-Catenin

Few fixed and permeabilized MCF-7 cells were applied to a reaction (20 μl) on ice, comprising 2 μl of 10×RT&T buffer (400 mM Tris HCl, pH 8.3 at 25° C., 350 mM KCl, 80 mM MgCl₂, and 100 mM DTE), 1 μM β-catenin-antisense promoter-linked primer 5′-dAAACGACGGC CAGTGAATTG TAATACGACT CACTATAGGC GCTCTGAAGA CAGTCTGTCG TGATGG-3′ (SEQ ID.15), 1 μM β-catenin-sense primer 5′-dATGGCAACCC AAGCTGACTT GATC-3′ (SEQ ID.16), ribonucleotide triphosphates (4 mM each for ATP GTP, CTP and UTP), deoxyribonucleotide triphosphates (4 mM each for dATP dGTP, dCTP and dTTP), and RNase inhibitors (10U). After C. therm./Taq DNA polymerase mixture (4U) was added, the reaction was incubated at 52° C. for 3 min, 65° C. for 30 min, 94° C. for 3 min, 52° C. for 3 min and then 68° C. for 3 min. A transcription reaction was prepared by adding T7 RNA polymerase (200U) and C. therm. polymerase (6U) mixture into above reaction. After three-hour incubation at 37° C., the resulting antisense RNA transcripts were continuously reverse-transcribed into sDNA-aRNA hybrids at 52° C. for 3 min and then 65° C. for 30 min. The quality of amplified sDNA-aRNA products can be assessed on a 1% formaldehyde-agarose gel (Lin (1999) supra). Above β-catenin sDNA-aRNA hybrid probe (10 μg) was treated by deaminase (10U, New England BioLab) for 30 min at 37° C. in 0.5×RT&T buffer. The resulting product was purified by microcon-30 filter, dissolved in 75 μl of Hepes buffer (pH 7.4).

### In-Cell Transfection and Gene Silencing Induction in MCF-7 Cancer Cells

Above β-catenin sDNA-aRNA hybrid probe (10 μg) in 75 μl of Hepes buffer (pH 7.4) was mixed with 50 μl of DOTAP liposome (1 mg/ml, Roche Biochemicals) on ice for 30 min before applied to 60 mm (2 ml) diameter culture dishes which contain 50% confluency of MCF-7 cancerous cells. The MCF-7 cells were grown in MEM medium with 10% bovine serum. After 72 hr incubation, the gene expression of β-catenin protein was shown by immuno-histochemical staining with 50 μg/ml anti-β-catenin antibodies (Santa Cruz BioLab) and found to be reduced more than 66˜70% in the sDNA-aRNA hybrid set while the blank and liposomal control sets have no significant gene silencing effects (FIG. 12(a)). The RNA-directed endoribonuclease (RDE) activity of the sDNA-aRNA hybrid transfection set was also detected to show a 167.2˜174.2% increase following the reduction of β-catenin expression (FIG. 12(b)). Such increase of RDE activity reflects a high RNAi effect induced by the sDNA-aRNA hybrid transfection. Because the over-expression of β-catenin oncogene has been known to increase the malignancy and metastasis of human breast cancers in vivo, the above findings could provide an effective therapy and/or anti-cancer drug for the prevention of cancer invasion and progression.

### Example 13

### Gene Silencing Using DNA-RNA Hybrids: Ex Vivo Model Targeting HIV-1 Genome in CD4⁺ Tc Lymphocyte Extracts

The foregoing establishes that the novel sDNA-aRNA hybrids of the present invention can be used in a novel strategy to knock out targeted gene expression in vitro. As discussed below, the novel sDNA-aRNA strategy of the invention is also effective in knocking out gene expression ex vivo.

As illustrated in the examples below, the methods and compositions of the invention are effective in knocking out exogenous viral gene expression ex vivo in a CD4⁺ Tc lymphocyte extract model. For molecules, HIV-1 genome from +1890 to +2230 bases was targeted because it has a critical role in viral replication activity, and for cells, CD4⁺ Tc lymphocyte was selected because it is a cell often targeted by HIV-1 infection. The HIV-1 is known to be the infectious pathogen of AIDS diseases. To a world-wide estimation till year 2000, more than 36 million people are currently infected by HIV-1, and this number is increased by at least 2 million per year. About four million AIDS patients have deceased this year due to the lack of an effective and stable long-term treatment for eradicating the malignancy of this virus.

The high mutation rate of HIV genome gradually generates more and more unexpected resistance to traditional HAART cocktail therapy, exacerbating the prevalence of this disease. Such dramatic increase of new mutant viruses as well as their carriers will soon become a very heavy finance burden for all health care and related disease prevention programs. However, although the high mutation rate of HIV-1 genome enable it to escape the traditional chemotherapy, it is impossible for HIV to change the whole targeted sequence which can be several hundred bases homologous to our sDNA-aRNA probe. Because the cosuppression effect of RNAi phenomenon to all homologous transcripts, the HIV genes is impossible to evade the silencing effects of sDNA-aRNA transfection by its mutations. It is very promising that the sDNA-aRNA transfection could become a powerful antiviral drug or vaccine for the prevention, or therapy, of viral infections.

FIG. 13(a) shows the gene silencing effect of anti-HIV-1 sDNA-aRNA transfections (n=3 for each set) in acute phase AIDS patient Tc lymphocyte extracts, while FIG. 13(b) shows the same effect in chronic phase AIDS patient Tc lymphocyte extracts. The lane 1 of FIG. 13(a) is pure HIV-1 genome to indicate the size location on an electrophoresis gel. The lane 2 of FIG. 13(a) and lane 1′ of FIG. 13(b) are Tc lymphocyte RNA extract samples from normal non-infected persons as negative control. The lane 3 of FIG. 13(a) and lane 2′ of FIG. 13(b) are extract samples from HIV-1-infected patients as positive control. In the acute phase (one-week infection), the treatment of 5 nM sDNA-aRNA transfection knocks out almost all viral gene expression, while those of 5 nM dsRNA and traditional antisense DNA transfection have very minor effects. In the chronic phase (two-year infection), the treatment of 25 nM sDNA-aRNA transfection knocks out 55.8% viral gene expression, while the transfections of 25 nM dsRNA and 250 nM traditional antisense DNA have no specific effects. When the sDNA-aRNA concentration is increased to 250 nM (FIG. 13(b), lane 6′), the transfection knocks out 61.3% viral gene expression without the induction of cytotoxicity. The expression of cellular house-keeping genes, GAPDH and β-actin, is normal and shows no interferon-induced non-specific RNA degradation in most of lanes, except the dsRNA treatments. These findings have directed to an immediate therapy potential for AIDS in both acute and chronic infections.

As discussed above, the experimental results establish that sDNA-aRNA hybrids potentially inhibit β-catenin expression in the MCF-7 cancer cells and also prevent HIV-1 viral activity in the CD4⁺ Tc lymphocytes. Thus, the results show that using a sDNA-aRNA duplex provides a powerful new strategy for gene therapy. At the highest dosage used in the experiments here (FIGS. 12 and 13), the sDNA-aRNA transfection did not cause interferon-induced cytotoxicity as previous reports in dsRNA transfections. This even underscores the fact that the sDNA-aRNA comprising compositions of the instant invention are effective even at low dosages. The results also indicate that this invention is effective in knocking out the targeted gene expression over a relatively long period of time. Further, it was observed that non-targeted cells appear to be normal, which implies that the compositions herein possess no overt toxicity. Thus, the invention offers the advantages of low dosage, stability, long term effectiveness, and lack of overt toxicity.

### Preparation of sDNA-aRNA Hybrid for Ex-Vivo Transduction and Gene Silencing of HIV

Partial human immunodeficiency virus-1 (HIV-1) genome sequence from +1760 to +3196 bases was cloned into pCR2.1 plasmid vector (Invitrogen) for the preparation of a sDNA-aRNA hybrid probe homologous to HIV-1 gag-pro-pol genes. Since the pCR2.1 plasmid contains a T7 promoter in front of its antisense clone site, the aRNA portion of the sDNA-aRNA hybrid construct can be directly amplified in an in-vitro transcription reaction (20 μl), comprising 21 μl of 10×RT&T buffer (400 mM Tris-HCl, pH 8.3 at 25° C., 300 mM KCl, 80 mM MgCl₂, 2M betaine, 100 mM DTE and 20 mM spermidine), rNTPs (4 mM each for ATP GTP, CTP and UTP), T7 RNA polymerase (200U), RNase inhibitors (10U) and the above pCR2.1 plasmid (10 pg). The reaction was performed at 37° C. for two hours and then reverse transcription (40 μl) was continuously performed in the same tube by adding 2 μl of 10×RT&T buffer, dNTPs (4 mM each for dGTP, dCTP, dTTP and 2 mM each for dATP and dITP), MMLV reverse transcriptase (30U) and 1 μM sense primer 5′-dGGATGICIGI CICCTTGTTG GTCC-3′ (SEQ ID.17). The reaction was further incubated at 37° C. for two hours, so as to provide about 30 μg sDNA-aRNA hybrid construct for transfection.

Above HIV-1 sDNA-aRNA hybrid probe (10 μg) was dissolved in 200 mM calcium phosphate and directly applied to 2 ml culture flask contain 50% confluency of CD4⁺ Tc lymphocytes. The Tc lymphocytes were extracted from patients and can be grown in human serum extracts with 100 μg/ml interleukin 2 (IL-2) for two weeks. After 96 hr incubation, the gene activity of HIV-1 genome was measured by Northern blotting and found to be almost completely shut down in the sDNA-aRNA hybrid transfection set (FIG. 13(a), lane 5; and FIG. 13(b), lanes 3′, 5′ & 6′). The blank control (FIG. 13(a), lane 2; and FIG. 13(b), lane 1′) and other construct transfection (FIG. 13(a), lanes 4 & 6; and FIG. 13(b), lanes 4′ & 7′) sets had no significant gene silencing effects. Unlike dsRNA treatment, the transfection of high concentrated sDNA-aRNA hybrids (250 nM; FIG. 13(b), lane 6′) did not cause any interferon-induced killing effects, because the house-keeping gene β-actin is normally expressed in all sets of transfected cells as well as non-transfected HIV-1-negative control (FIG. 13(a), lane 2; and FIG. 13(b), lane 1′) and -positive (FIG. 13(a), lane 3; and FIG. 13(b), lane 2′) control sets. The FIG. 13(a) showed the acute transfection results of HIV-1 sDNA-aRNA hybrids in one-week-infection patients, while the FIG. 13(b) showed the chronic transfection results of HIV-1 sDNA-aRNA hybrids in two-year-infection patients. Because the Northern blot method is able to detect HIV-1 gene transcript at the nanogram level, the above strong viral gene silencing effect actually demonstrates a very promising pharmaceutical and therapeutical use of this sDNA-aRNA hybrid construct as antiviral drugs and/or vaccines.

### Example 14

### Gene Silencing Using sDNA-aRNA Hybrids: In Vivo Model: Interfering Tyrosinase Gene Expression in Mouse Skin Hairs

The foregoing establishes that the novel sDNA-aRNA hybrids of the present invention can be used in a novel strategy to knock out targeted gene expression in vitro as well as ex vivo. As discussed below, the novel sDNA-aRNA strategy of the invention is also effective in knocking out gene expression in vivo.

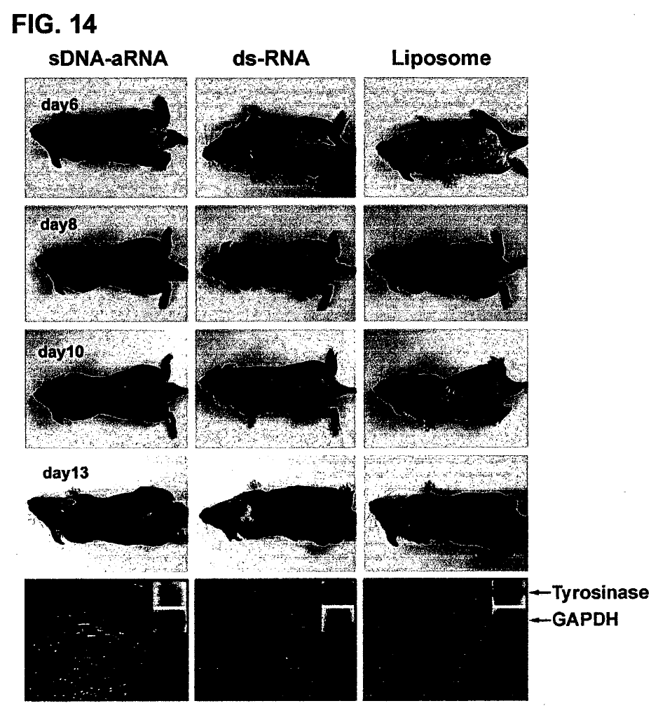

As illustrated in the examples below, the methods and compositions of the invention are effective in knocking out specific gene expression in vivo in a mouse skin hair model. As shown in FIG. 14, albino (white) skin hairs of melanin-knockout mice were created by four times of intra-cutaneous (i.c.) transduction of about 50 nM mismatched sDNA-aRNA per day against tyrosinase (tyr) gene transcripts. The expression of melanin (black pigment) in skins and hairs has been blocked due to a loss of its intermediate generation by the tyrosinase knockout. Contrarily, the control and double-stranded RNA (dsRNA) transfected mice presented normal skin color (black), indicating that the loss of melanin is specific to RNAi silencing effect induced by the sDNA-aRNA transfection. Moreover, Northern blotting showed a 76.1±5.3% reduction of tyr gene expression after the sDNA-aRNA transfection, while minor non-specific degradation of common gene transcripts (such as GAPDH) was detected in the dsRNA transfected skins.

As discussed here, the experimental results establish that sDNA-aRNA hybrids potentially inhibit tyrosinase gene expression in the transfected mice skins and therefore prevent the production of melanin (black pigment) in hairs. Thus, the results show that using a sDNA-aRNA duplex provides a powerful new strategy for gene therapy, especially to melanoma. At the same dosage (200 nM in total), the sDNA-aRNA transfection did not cause any cytotoxicity effect, while the dsRNA transfections induced detectable non-specific mRNA degradation as previous reports (Stark (1998) supra, and Elbashir (2001) supra). This even underscores the fact that the sDNA-aRNA comprising compositions of the instant invention are effective even under in vivo systems without the side-effects of dsRNA. The results also indicate that this invention is effective in knocking out the targeted gene expression over a relatively long period of time because the hair regrowth requires at least ten-day recovery. Further, it was observed that non-targeted skin hairs appear to be normal, which implies that the compositions herein possess high specificity and no overt toxicity. Thus, the invention offers the advantages of low in-vivo dosage, stability, long term effectiveness, and lack of overt toxicity.

### Preparation of sDNA-aRNA Hybrid for In-Vivo Transduction and Gene Silencing in Mouse

Partial Mus musculus tyrosinase (tyr) sense DNA (sDNA) sequence (SEQ ID. 18) purchased from a core facility (Invitrogen) was synthesized by an oilgonucleotide synthesizer machine. The complementary antisense RNA (aRNA) sequence (SEQ ID. 19) was transcribed from a tyr-inserted RCAS-viral vector which is a genetically engineered retrovirus capable of delivering a gene insert of interest or its related components into a host cell genome and expressing the gene products, such as RNA, peptide and protein in the cell. The synthesized sDNA was boiled at 94° C., 10 min in diethyl pyrocarbonate (DEPC)-added H₂O (˜pH 5.5) for partial deamination. Such deamination will introduce some mismatched base pairs in a sDNA-aRNA hybrid. Hybridization of the tyr sDNA and aRNA was accomplished by incubation of 200 μg of each sequence in a 20 mM Hepes buffer (pH 6.5) at 68° C. for over 10 min and then gradually cooling from 50° C. to 10° C. over a period of 30 min. The final sDNA-aRNA product was stored in a −80 freezer before used.

The dorsal hairs of one-month-old W-9 black mice were stripped by wax. Four intra-cutaneous injections of the tyr sDNA-aRNA (25 μg for each injection) were applied by a 24 hr interval fashion for each injection. After a thirteen-day hair regrowth period, white hairs were observed only in the injected area of the sDNA-aRNA transfected mice, while those of the dsRNA transfected and blank control mice showed normal black colored hairs. Northern analysis of the tyr gene expression indicated a 76.1±5.3% reduction in the transfected skins of the sDNA-aRNA treated mice, but no such gene silencing effect was found in the dsRNA transfected and blank control mice.

### Example 15

The sDNA-aRNA hybrid molecule can be used for a wide variety of applications in relation to inducing RNA interference or altering the characteristic of the cell. In one example, the DNA-RNA hybrid molecule may serve as a therapeutic agent to effectuate a therapeutically desirable outcome in physiological conditions. As seen in examples 1-3, the DNA-RNA may be used to inhibit proliferation of cancer cells, fight viral infection, and alter pigmentation of cells. Although the examples above used a single species of DNA-RNA hybrid molecule for each condition, it is contemplated that multiple species of DNA-RNA hybrids, as a cocktail, may be used to combat multi-factorial disease condition. Preferably, the cocktail may include at least two to ten species of DNA-RNA hybrid each having a different nucleic acid sequence from the other species in the cocktail.

In order for the DNA-RNA hybrid to be effectively delivered into the cell in vivo as a therapeutic agent, it is also contemplated that DNA-RNA hybrid molecules or multiple species of the molecule will be formulated for effective delivery. Examples of formulations may include formulations in saline, liposomes such as DOTAP and colipids, polymers such as polyethylene glycol, polyvinyl pyrrolidone, poly vinyl alcohol, and other transfection inducing polymers and agents. Delivery may be via intramuscular, intra-dermal, intra-tumor, intraperitoneal, systemic injections with or without electroporation. The formulations may also include targeting ligands such as antibodies specific to a particular cell to form, for example, immunoliposomes. Preferably, the therapeutic formulation of the DNA-RNA hybrid molecule would be contained in an article of manufacture such as a kit, bottle, or tube with a label indicating its use. The label may be affixed to the article or may be separate such as an instruction sheet or manual.

In certain situations, it is also contemplated that the DNA-RNA would not need to be delivered into the body. For example, ex vivo inhibition of viral replication such as disclosed in Example 2 may be useful. The HIV patients may have their own CD4⁺, or any other blood, marrow, or precursor cells, removed and treated and transplanted back into their system. This eliminates any immune type rejection of the transplanted cells that would have occur if the cells were from a different individual.

In the area of specialized individual medicine, the DNA-RNA hybrid molecule may also be used to treat individualized conditions. For example, a differential expression analysis using microarray or subtractive hybridization technology may be used to determine aberrant gene expression in an individual having the condition to be treated. Overexpressing genes, such as seen in cancer cells overexpressing β-catenin, may then be knock out or down by the generating DNA-RNA hybrid molecules directed toward those genes. A kit may be provided to the treating physician for performing the subtractive hybridization and using the over-expressed genes remaining from the subtraction as templates for DNA-RNA interference.

In another example, gene function may be analyzed for unknown or known genes by impairing its expression using the DNA-RNA hybrid molecule just described. As genes are identified from the Human Genome Project, DNA-RNA hybrid molecules may be generated and transfected into cells to observe the effect of the impairment of expression in the cell. As such, the function of the gene impaired may be deduced. The DNA-RNA hybrid may be introduced into the cell by a number of different methods such as micro-injection, electroporation, transfection by liposomes, calcium phosphate, dextran sulfates, or polymers.

In one specific embodiment, the method of the present invention comprises the steps of: a) providing: i) a substrate expressing a targeted gene, and ii) a composition comprising a DNA-RNA hybrid capable of silencing the expression of the targeted gene in the substrate; b) treating the substrate with the composition under conditions such that gene expression in the substrate is inhibited. The substrate can express the targeted gene in vitro or in vivo.

In another specific embodiment, the method for inducing gene silencing effects using DNA-RNA hybrid constructs comprises the steps of:

a. providing a plurality of DNA sequences, wherein said DNA sequences are homologous to a or a plurality of targeted intracellular messenger RNA sequences;

b. contacting said DNA sequences to a plurality of RNA sequences to form a plurality of DNA-RNA hybrids, wherein said RNA sequences are complementary to said DNA and intracellular messenger RNA sequences; and

c. transducing said DNA-RNA hybrids into a plurality of cells which are sensitive to RNA interference effects; and so as to provide a specific gene silencing effect to the targeted messenger RNAs within said cells.

The said DNA sequences may be synthesized by a machine such as an oligonucleotide synthesizer, a thermocycler, an isothermal incubator, or any other suitable machine for synthesizing DNA sequences. Preferably, the said DNA sequences are form from one or a plurality of nucleic acid templates using enzymatic reaction such as reverse transcription, polymerase chain reaction, nucleic acid sequence based amplification, and RNA-polymerase cycling reaction. The templates may be single or double stranded, linear of circular structures. For purpose of gene silencing, the said DNA sequences may be completely or partially homologous to said intracellular messenger RNA sequences that are targeted.

Similarly, the said RNA sequences may be synthesized by a machine such as an oligonucleotide synthesizer, thermocycler or isothermal incubator. The RNA, preferably, may be generated from one or a plurality of nucleic acid templates by enzymatic methods such as in-vitro transcription, aRNA amplification, nucleic acid sequence based amplification, and RNA-polymerase cycling reaction. The templates may also be single or double stranded, linear or circular in structures. The said RNA sequences may be completely or partially complementary to said DNA sequences.

Synthesis of the RNA and DNA molecules may also be performed separately and allowed to anneal or hybridize to form duplex sequences. Preferably the hybridization occurs in a Hepes-containing buffer at about 68° C. for more than 10 minutes. The Hepes-containing buffer is preferably a 20 mM HEPES solution.

In a further embodiment, a kit is provided for inducing gene silencing effects using DNA-RNA hybrid constructs. The kit comprises the following components:

a. a plurality of DNA-RNA hybrid constructs, wherein the DNA portion of said DNA-RNA hybrid constructs are homologous to a or a plurality of targeted intracellular messenger RNA sequences; and

b. a plurality of transfection reagents, wherein said transfection reagents can deliver said DNA-RNA hybrid constructs into a plurality of targeted cells; and so as to provide a specific gene silencing effect to the targeted messenger RNAs within said cells.

### Example 16

Another embodiment of the present invention is the modification of the RNA-Polymerase Chain Reaction (RNA-PCR) as disclosed in U.S. Pat. No. 6,197,554 having common inventors in this application. The modification being the use of primers having sequence specific sequences and the RNA promoter sequences to amplify and generate DNA-RNA hybrid molecules.

Briefly, the elevated thermocycling temperature of the RNA-PCR method prevents rapid degradation of short-lived RNAs and further reduces the secondary structure of RNAs to increase the accessibility of enzyme interactions and the production of more complete desired RNAs. The procedure uses thermostable enzymes, including Tth-like polymerases with reverse transcriptase activity. The use of proofreading RNA polymerases for amplification not only provides higher fidelity but also eliminates preferential amplification of abundant RNA species. Additionally, rapid and simple cell fixation and permeabilization steps inhibit any alterations in gene expression during specimen handling or genomic contamination. (See, Embleton et al., (1992) Nucl. Acids Res. 20, 3831-3837).

In yet another embodiment, the method for generating DNA-RNA hybrids for gene silencing comprises the steps of:

a. providing: i) a solution comprising a nucleic acid template, ii) one or more primers sufficiently complementary to the sense conformation of the nucleic acid template, and iii) one or more promoter-linked primers sufficiently complementary to the antisense conformation of the nucleic acid template, and having an RNA promoter;

b. treating the nucleic acid template with one or more primers under conditions such that a first DNA strand is synthesized;

c. treating the first DNA strand with one or more promoter-linked primers under conditions such that a promoter-linked double-stranded nucleic acid is synthesized;

d. treating the promoter-linked double-stranded nucleic acid under conditions such that essentially RNA fragments are synthesized; and

e) treating RNA fragments with one or more primers under conditions such that a DNA-RNA hybrids are synthesized.

Steps b) through e) of the above method are preferably repeated for a sufficient number of cycles to obtain a desired amount of amplified hybrid product. Step b), for example, may include heating the solution at a temperature above 90° C. to provide denatured nucleic acids. Step c), for example may include treating the first DNA strand with one or more promoter-linked primers at a temperature ranging from about 37° C. to about 72° C. in the presence of a plurality of polymerases. Examples of the polymerases include DNA-dependent DNA polymerases, RNA-dependent DNA polymerases, RNA polymerases, Taq-like DNA polymerase, Tth-like DNA polymerase, C. therm. polymerase, viral replicases, and combinations thereof. The viral replicases can be selected from the group consisting of Avian myeloblastosis virus (AMV) reverse transcriptase and Moloney murine leukemia virus (MMLV) reverse transcriptase, Bromo mosaic virus (BMV) replicase and derivatives of reverse transcriptases that do not have RNase H activity. Step d) may include treating the promoter-linked double-stranded nucleic acid with an enzyme having transcriptase activity at about 37° C. such as T3 RNA polymerase, T7 RNA polymerase, SP6 RNA polymerase, and M13 RNA polymerase. Step d) may also include treating the promoter-linked double-stranded nucleic acid with viral replicases such as AMV reverse transcriptase, MMLV reverse transcriptase, BMV replicase and derivatives of reverse transcriptases that do not have RNase H activity.

In another embodiment, the method of improved RNA-polymerase cycling reaction which amplifies a specific DNA-RNA hybrid construct for transducing biological gene silencing effects comprises the steps of:

a. providing a plurality of nucleic acid sequences as an amplifiable gene template for following reactions;

b. denaturing and contacting said nucleic acid template with a plurality of primers and a plurality of promoter-linked primers, wherein said primers and promoter-linked primers are respectively complementary to the sense and antisense sequence conformation of said nucleic acid template;

c. permitting extension of said primers and promoter-linked primers to form a plurality of promoter-linked double-stranded nucleic acid sequences, wherein said promoter-linked double-stranded nucleic acid sequences are formed by either DNA-directed or RNA-directed DNA and/or RNA polymerases or the combination thereof;

d. permitting transcription of said promoter-linked double-stranded nucleic acid sequences to form a plurality of amplified RNA fragments, wherein said amplified RNA fragments are generated by extension of RNA polymerase activity through the promoter region of said promoter-linked double-stranded DNAs; and

e. contacting said amplified RNA fragments with said primer to form a plurality of DNA-RNA hybrid duplexes, wherein said DNA-RNA hybrid duplexes are formed by reverse transcription of said amplified RNA fragments with the extension of said primer; so as to provide amplified sDNA-aRNA hybrids ready for inducing RNAi-related gene silencing effects.

To increase the yield of sDNA-aRNA hybrids, steps (b) through (e) may be repeated at least one time. Furthermore, it may be preferable to have a plurality of nucleotide analogs into the sDNA part of said amplified sDNA-aRNA hybrids in the step (e) to increase the onset of gene silencing effects. The nucleotide analogs by be generated by treatment with deaminase or chemical treatments such as using acidic solutions. With respect to the denaturing step in step b), it is preferred to use temperature at a range from about 90° C. to about 100° C., while the enzyme activities are preferably performed at temperature ranging from 37° C. to about 70° C.

### REFERENCES

The following references are hereby incorporated by reference as if fully set forth herein:

1. Grant et. al., “Dissecting the mechanisms of posttranscriptional gene silencing: divide and conquer”, Cell 96, 303-306 (1999).

2. Kennerdell, J. R. and Carthew, R. M., Cell 95, 1017-1026 (1998).

3. Misquitta, L. and Paterson, B. M. (1999) Proc. Natl. Acad. Sci. USA 96, 1451-1456.

4. Pal-Bhadra, M., Bhadra, U., and Birchler, J. A. (1999) Cell 99, 35-46.

5. Tabara, H., Sarkissian, M., Kelly, W. G., Fleenor, J., Grishok, A., and Timmons, L. (1999) Cell 99, 123-132.

6. Ketting, R. F., Haverkamp, T. H., van Luenen, H. G., and Plasterk, R. H. (1999) Cell 99, 133-141.

7. Fire, A., Xu, S., Montgomery, M. K., Kostas, S. A., Driver, S. E., and Mello, C. C., “Potent and specific genetic interference by double-stranded RNA in Caenorhabditis elegans”, Nature 391: 806-811 (1998).

8. Grishok, A., Tabara, H., and Mello, C. C. (2000) Science 287, 2494-2497.

9. Wargelius, A., Ellingsen, S., and Fjose, A. (1999) Biochem. Biophys. Res. Commun. 263, 156-161.

10. Wianny, F. and Zemicka-Goetz, M. (2000) Nature Cell Biol. 2, 70-75.

11. Bosher, J. M. and Labouesse, M., “RNA interference: genetic wand and genetic watchdog”, Nature Cell Biology 2: 31-36 (2000).

12. Zamore, P. D., Tuschl, T., Sharp, P. A., and Bartel, D. P. (2000) Cell 101, 25-33.

13. Yang, D., Lu, H., and Erickson, J. W. (2000) Current Biology 10, 1191-1200.

14. Cogoni, C. and Macino, G. (1999) Nature 399, 166-169.

15. Smardon, A., Spoerke, J. M., Stacey, S. C., Klein, M. E., Mackin, N., and Maine, E. M. (2000) Curr. Biol. 10, 169-171.

16. Raffo, A. J., Perlman, H., Chen, M. W., Day, M. L., Streitman, J. S., and Buttyan, R. (1995) Cancer Res. 55, 4438-4445.

17. Colombel, M., Symmans, F., Gil, S., O'Toole, K. M., Chopin, D., Benson, M., Olsson, C. A., Korsmeyer, S., Buttyan, R. (1993) Am. J. Pathol. 143, 390-400.

18. Berchem, G. J., Bosseler, M., Sugars, L. Y., Voeller, H. J., Zeitlin, S., and Gelmann, E. P. (1995) Cancer Res. 55, 735-738.

19. McConkey, D. J., Greene, G., and Pettaway, C. A. (1996) Cancer Res. 56, 5594-5599.

20. Elbashir et. al., “Duplexes of 21-nucleotide RNAs mediate RNA interference in cultured mammalian cells”, Nature 411, 494-498 (2001).

21. Stark et. al., “How cells respond to interferons”, Annu. Rev. Biochem. 67, 227-264 (1998).

22. Bahramian et. al., “Transcriptional and posttranscriptional silencing of rodent α1(I) collagen by homologous transcriptionally self-silenced transgene”, Mol. Cell. Biology 19, 274-283 (1999).

23. Shi-Lung Lin, Cheng-Ming Chuong, Randall B. Widelitz and Shao-Yao Ying, “In vivo analysis of cancerous gene expression by RNA-polymerase chain reaction”, Nucleic Acid Res. 27, 4585-4589 (1999).

24. Alexeev et. al., “Localized in vivo genotypic and phenotypic correction of the albino mutation in skin by RNA-DNA oligonucleotide”, Nat. Biotechnol. 18, 43-47 (2000).

25. Shi-Lung Lin, Cheng-Ming Chuong and Shao-Yao Ying, “A novel mRNA-cDNA interference phenomenon for silencing bcl-2 expression in human LNCaP cells”, Biochem. Biophys. Res. Commun. 281, 639-644 (2001).

26. Shi-Lung Lin and Shao-Yao Ying, “D-RNAi (Messenger RNA-antisense DNA Interference Phenomenon) is a novel defense system against cancers and viral infections”, Current Cancer Drug Targets 1, 241-247 (2001).

27. Sambrook et. al., “Molecular Cloning, 2^{nd} Ed.”, Cold Spring Harbor Laboratory Press, pp8.11-8.19 and pp 7.39-7.52 (1989).

28. Compton, J., “Nucleic acid sequence-based amplification”, Nature 350, 91-92 (1991).

29. Kacian et al., Proc. Natl. Acad. Sci. USA 69, 3038-3044 (1972).

30. Chamberlin et al., Nature 228, 227-231 (1970).

31. Myers and Gelfand, Biochemistry 30, 7662-7666 (1991).

32. Scott W. Knight and Brenda L. Bass, “A role for the RNase III enzyme DCR-1 in RNA interference and germ line development in Caenorhabditis elegans”, Science 293, 2269-2271 (2001).

33. Kwoh et al., Proc. Natl. Acad. Sci. USA 86, 1173-1177 (1989).

34. Embleton et al., Nucl. Acids Res. 20, 3831-3837 (1992).

35. Hannoush et. al., J. Am. Chem. Soc. 123, 12368-12374 (2001).

36. U.S. Pat. No. 4,683,195 issued to Mullis et. al.

37. U.S. Pat. No. 4,683,202 issued to Mullis et. al.

38. U.S. Pat. No. 4,965,188 issued to Mullis et. al.

39. U.S. Pat. No. 5,075,216 issued to Innis et. al.

40. U.S. Pat. No. 5,322,770 issued to Gelfand et. al.

41. U.S. Pat. No. 5,817,465 issued to Mallet et. al.

42. U.S. Pat. No. 5,888,779 issued to Kacian et. al.

43. U.S. Pat. No. 6,197,554 issued to Shi-Lung Lin et. al.

44. U.S. Pat. No. 6,130,040 issued to Shi-Lung Lin et. al.

45. U.S. Pat. No. 5,795,715 issued to Livache et. al.

46. Patent Cooperation Treaty Publication No. WO 00/75356 issued to Lin et. al.

47. U.S. Pat. No. 4,289,850 issued to Robinson.

48. U.S. Pat. No. 6,159,714 issued to Lau.

49. U.S. Pat. Nos. 4,945,082, 4,950,652, 5,091,374 and 5,906,980 issued to Carter.

It is understood that the examples and embodiments described herein are for illustrative purposes only and that various modifications or changes in light thereof will be suggested to persons skilled in the art, and are to be included within the spirit and purview of the invention as set forth in the appended claims. All publications and patents cited herein are incorporated herein by reference in their entirety for all purposes.

What is claimed is:

1. A method for generating DNA-RNA hybrid constructs, comprising the steps of:
(a) providing: i) a solution comprising a nucleic acid template, ii) one or more primers sufficiently complementary to one oriented conformation of said nucleic acid template, and iii) one or more promoter-linked primers sufficiently complementary to the reversely oriented conformation of said nucleic acid template, and having an RNA promoter;
(b) treating said nucleic acid template with said one or more primers under conditions such that a first DNA strand is synthesized; (c) treating said first DNA strand with said one or more promoter-linked primers under conditions such that a promoter-linked double-stranded nucleic acid is synthesized; (d) treating said promoter-linked double-stranded nucleic acid under conditions such that essentially amplified RNA fragments are synthesized; and (e) treating said RNA fragments with said one or more primers under conditions such that DNA-RNA hybrids are synthesized by reverse transcription of said amplified RNA fragments with the extension of said one or more primers.

2. The method of claim 1 further comprising the step of repeating steps b) through e) for a sufficient number of cycles to obtain a desired amount of amplified product.

3. The method of claim 1, wherein said treating step in step b) comprises heating said solution at a temperature above 90° C. to provide denatured nucleic acids.

4. The method of claim 1, wherein said treating step in step c) comprises pre-treating said first DNA strand with said one or more promoter-linked primers at a temperature ranging from about 35° C. to about 75° C.

5. The method of claim 1, wherein said treating step in step c) comprises treating said DNA strand with one or more promoter-linked primers in the presence of a polymerase.

6. The method of claim 5, wherein said polymerase is selected from the group consisting of DNA-dependent DNA polymerases, RNA-dependent DNA polymerases, RNA polymerases, Taq-like DNA polymerase, Tth-like DNA polymerase, C. therm. polymerase, viral replicases, and combinations thereof.

7. The method of claim 6, wherein said viral replicases are selected from the group consisting of avian myeloblastosis virus reverse transcriptase and Moloney murine leukemia virus reverse transcriptase, Brome mosaic virus replicase, Trichomonas vaginalis virus replicase, Flock house virus replicase, Q beta replicase, and mutants or combinations thereof.

8. The method of claim 7, wherein said avian myeloblastosis virus reverse transcriptase does not have RNase H activity.

9. The method of claim 1, wherein said treating step in step d) comprises treating said promoter-linked double-stranded nucleic acid with an enzyme having transcriptase activity at about 37° C.

10. The method of claim 9, wherein said enzyme having transcriptase activity is selected from the group consisting of T3 RNA polymerase, T7 RNA polymerase, SP6 RNA polymerase, M13 RNA polymerase and viral replicase.

11. The method as defined in claim 1, wherein said treating step in step e) comprises pre-treating said RNA fragments with said one or more primers at a temperature ranging from about 37° C. to about 72° C.

12. The method of claim 1, wherein said one or more promoter-linked primers are complementary to the 3′-ends of the antisense conformation of said nucleic acid template when said one or more primers are complementary to the 3′-ends of the sense conformation of said nucleic acid template.

13. The method of claim 1, wherein said one or more promoter-linked primers are complementary to the 3′-ends of the sense conformation of said nucleic acid template when said one or more primers are complementary to the 3′-ends of the antisense conformation of said nucleic acid template.

14. The method of claim 1, further comprising the step of generating one or more mismatched nucleotides in said DNA-RNA hybrid for gene silencing induction.

15. The method of claim 14, wherein said mismatched nucleotides are generated by enzymes selected from the group consisting of deaminase, Taq-like DNA polymerase, Tth-like DNA polymerase and viral replicases, or other low fidelity enzymes.

16. The method of claim 14, wherein said mismatched nucleotides are generated by chemical modification selected from the group consisting of weak acids, mild acetic anhydride and machine incorporation.

17. The method of claim 14, wherein said mismatched nucleotides are selected from the group consisting of deoxyuracil, inosine, xanthine, hypoxanthine, labeled nucleotide, ribonucleotide in a DNA construct, deoxyribonucleotide in an RNA construct, 7-deaza-dNTP, methylthio-linked nucleotide, phosphothio-linked nucleotide, morpholino nucleotide, peptide nucleic acid (PNA), and viral genome nucleic acid, etc.

18. The method of claim 1, further comprising the step of incorporating one or more nucleotide analogs into said DNA-RNA hybrid to increase gene silencing induction or to stabilize gene silencing effects.

19. The method of claim 18, wherein said nucleotide analogs are selected from the group consisting of deoxyuracil, labeled nucleotide, ribonucleotide in the DNA construct, deoxyribonucleotide in the RNA construct, 7-deaza-dNTP, methylthio-linked nucleotide, phosphothio-linked nucleotide, morpholino nucleotide, hexose-containing nucleotide, peptide nucleic acid (PNA) and their derivatives.

20. The method of claim 1, further comprising the step of contacting said DNA-RNA hybrid with a reagent for transfecting a eukaryotic cell for inhibiting the expression of at least one gene.

21. The method of claim 20, wherein said reagent is selected from the group consisting of chemical transfection reagents and liposomal transfection reagents.

22. The method as defined in claim 20, wherein said gene comprises a gene selected from the group consisting of pathogenic nucleic acids, viral genes, mutated genes, oncogenes and unknown functional genes.

23. A composition for inhibiting the expression of at least one targeted gene in a substrate, the composition comprising: a DNA-RNA hybrid.

24. The composition of claim 23, wherein the DNA-RNA hybrid is synthesized using the method of claim 1.

25. The composition of claim 23, wherein the RNA of said DNA-RNA hybrid is comprised of either part or all of the spliced mRNA transcript of the targeted gene.

26. The composition of claim 23, wherein the RNA of said DNA-RNA hybrid is either homologous or complementary to part or all of the unspliced mRNA transcript of the targeted gene.

27. The composition of claim 23, wherein the RNA of said DNA-RNA hybrid is either homologous or complementary to the combination of part or all of the unspliced and spliced mRNA transcript of the targeted gene.

28. The composition of claim 23, wherein the DNA-RNA hybrid is made by complementarily combining the RNA molecule of claims 25 or 26 with its corresponding complementary DNA molecule in a base-paring double-stranded form.

29. The composition of claim 28, wherein the complementary RNA and DNA molecules are synthetic nucleotide sequences.

30. The composition of claim 23, wherein the substrate is a cell or an organism.

31. The composition of claim 23, further comprising a carrier molecule, which carrier molecule is capable of being taken up by a cell.

32. A method for inhibiting the expression of a targeted gene in a substrate that expresses the targeted gene, comprising the steps of
a) providing a composition comprising a DNA-RNA hybrid capable of inhibiting the expression of said targeted gene in said substrate; and
b) contacting said substrate with said composition under conditions such that the expression of said gene in said substrate is inhibited.

33. The method of claim 32, wherein said composition is the composition of claim 24 or claim 28 or both.

34. The method of claim 32, wherein said substrate expresses said targeted gene in vivo.

35. The method of claim 32, wherein said targeted gene comprises a gene selected from the group consisting of pathogenic nucleic acids, viral genes, mutated genes, oncogenes and unknown functional genes.

36. The method of claim 32, wherein said DNA-RNA hybrid inhibits β-catenin oncogene expression.

37. The method of claim 32, wherein said DNA-RNA hybrid inhibits bcl-2 drug-resistant gene expression.

38. The method of claim 32, wherein said substrate is a prokaryote.

39. The method of claim 38, wherein said prokaryote is a virus.

40. The method of claim 38, wherein said prokaryote is a bacterial cell.

41. The method of claim 32, wherein said substrate is an eukaryote or the cell of said eukaryote.

42. The method of claim 41, wherein said eukaryote is a vertebrate.

43. The method of claim 41, wherein said eukaryote is a mouse or rat.

44. The method of claim 41, wherein said eukaryote is a chimpanzee.

45. The method of claim 41, wherein said eukaryote is a human being.

46. An isolated nucleic acid molecule comprising a first strand of deoxynucleic acid (DNA) coupled to a second strand of riboxynucleic acid (RNA), wherein the RNA comprises nucleic acid sequence that is either homologous to or complementary to a messenger RNA (mRNA) molecule.

47. The isolated nucleic acid molecule of claim 46 comprises no nucleotide analog.

48. The isolated nucleic acid molecule of claim 46 comprises at least one nucleotide analog which is selected from the group consisting of inosine, xanthine, hypoxanthine, deoxyuracil, ribonucleotide in a DNA linkage, deoxyribonucleotide in an RNA linkage, 7-deaza-dNTP, labeled nucleotides, and their derivative analogs.

49. The isolated nucleic acid molecule of claim 48 wherein the derivative of said nucleotide analog is preferably selected from the group consisting of hexose-containing, 2′-5′ linked, phosphothio-linked, methylthio-linked, morpholino-linked and peptide-linked nucleotide analogs.

50. The isolated nucleic acid molecule of claim 48 wherein the nucleotide analog is a depurinated nucleotide.

51. The isolated nucleic acid molecule of claim 46, wherein the DNA and RNA are at least 20 percent complementary.

52. The isolated nucleic acid molecule of claim 51, wherein the DNA and RNA are about 95 percent complementary when the RNA comprises at least one palindromic sequence.

53. The isolated nucleic acid molecule of claim 52, wherein the RNA is at least about 45 percent complementary to the targeted mRNA.

54. The isolated nucleic acid molecule of claim 46, wherein the DNA comprises SEQ ID NO: 2.

55. The isolated nucleic acid molecule of claim 46 wherein the DNA comprises SEQ ID NO: 3.

56. The isolated nucleic acid molecule of claim 46 wherein the DNA comprises SEQ ID NO: 4.

57. The isolated nucleic acid molecule of claim 46 wherein the mRNA is expressed from a gene of interest.

58. The isolated nucleic acid molecule of claim 57 wherein the gene of interest is an oncogene.

59. The isolated nucleic acid molecule of claim 58, wherein the oncogene is β-catenin.

60. The isolated nucleic acid molecule of claim 57, wherein the gene of interest is a viral gene or genome.

61. The isolated nucleic acid molecule of claim 60, wherein the viral genome is a DNA or RNA molecule containing partial or full of the viral genome.

62. The isolated nucleic acid molecule of claim 61, wherein the viral genome is a plurality of viral genes from the HIV-1 genome ranging from about +1890 to +2230 bases.

63. The isolated nucleic acid molecule of claim 46, wherein the gene of interest expresses a protein.

64. The isolated nucleic acid molecule of claim 63, wherein the protein is tyrosinase.

65. The isolated nucleic acid molecule of claim 52, wherein the nucleic acid sequence of the RNA is at least about 48% complementary to the corresponding portion of the messenger riboxynucleic acid molecule.

66. The isolated nucleic acid molecule of claim 23 or claim 46, wherein the nucleic acid molecule is double stranded nucleotide sequences ranging from about 20 to about 10,000 basepairs.

67. The isolated nucleic acid molecule of claim 23 or claim 46, wherein the double stranded nucleic acid molecule is sized ranging from about 20 to about 150 basepairs.

68. The isolated nucleic acid molecule of claim 23 or claim 46, wherein the DNA comprises at least one labeled deoxyribonucleotide.

69. The isolated nucleic acid molecule of claim 23 or claim 46, wherein the labeled ribonucleotide is labeled with a molecule selected from the group consisting of a fluorophore, a hapten, a ligand, an enzyme, and a radioactive molecule.

70. The use of the isolated nucleic acid molecule of claim 23 or claim 46 to alter the characteristic of an eukaryotic cell.

71. The use of claim 23 or claim 46 wherein the characteristic is selected from the group consisting of (a) expression of a protein; (b) cell division rate; (c) pigmentation.

72. The use of claim 23 or claim 46 wherein the isolate nucleic acid molecule has an effect that lasts at least three days.

73. The use of the isolated nucleic acid molecule of claim 23 or claim 46 to inhibit the expression of messenger RNA in a cell.

74. The use of claim 23 or claim 46 wherein the messenger RNA is transcribed from a gene selected from a group consisting of viral gene, oncogene, enzyme.

75. The use of claim 23 or claim 46 wherein the isolated nucleic acid molecule is used at a concentration ranging from about 1 nM to about 750 nM.

76. The use of claim 23 or claim 46 wherein the isolated nucleic acid molecule, wherein the concentration ranges from about 5 nM to about 50 nM.

77. A composition comprising the isolated nucleic acid molecule of claim 23 or claim 46 and a transfection agent.

78. The composition of claim 23 or claim 46 wherein the transfection agent is selected from the group consisting of saline solution, calcium phosphate, liposomes, lipid derivatives, dextran sulfate, and polymers.

79. A composition comprising multiple species of the isolated nucleic acid molecule of claim 23 or claim 46, wherein each species has a different nucleic acid sequence than another.

80. The composition of claim 23 or claim 46 wherein the number of species ranges from two to ten.

81. An article of manufacture comprising a container comprising the isolated nucleic acid of claim 23 or claim 46 and a label providing information on the use of the isolated nucleic acid of claim 23 or claim 46.

82. The article of manufacture of claim 23 or claim 46 wherein the label is affixed to the container.

83. The article of manufacture of claim 23 or claim 46 wherein the label is an instruction sheet or an instruction manual.

84. A method of making a DNA-RNA hybrid molecule capable of altering the characteristic of an eukaryotic cell, the method comprising the steps of:
a) synthesizing an RNA molecule with a sequence either homologous or complementary to a RNA species in a cell;
b) synthesizing a DNA molecule with a sequence complementary to the RNA molecule of (a);
c) forming a DNA-RNA hybrid molecule from the RNA molecule of (a) and the DNA molecule of (b); wherein the DNA-RNA hybrid molecule is capable of altering the characteristic of the cell.

85. The method of claim 84 wherein the hybrid comprises at least one nucleotide analog.

86. The method of claim 85 wherein the at least one nucleotide analog is selected from the group consisting of inosine, xanthine, hypoxanthine, deoxyuracil, ribonucleotide in a DNA linkage, deoxyribonucleotide in an RNA linkage, 7-deaza-dNTP, labeled nucleotides, and their derivatives which are preferably selected from the group consisting of hexose-containing, 2′-5′ linked, phosphothio-linked, methylthio-linked, morpholino-linked and peptide-linked nucleotide analogs.

87. The method of claim 84 further comprising the step of treating the DNA molecule with deaminase.

88. The method of claim 84, wherein the DNA and RNA are at least 45% complementary.

89. The method of claim 84 wherein the step of synthesizing DNA molecule is by chemical synthesis.

90. The method of claim 84 wherein the step of synthesizing the DNA molecule is by reverse transcription from an RNA molecule.

91. The method of claim 84 wherein the step of synthesizing the DNA molecule is by polymerase chain reaction from the a DNA molecule.

92. The method of claim 84 wherein the DNA molecule is a viral genome.

93. The method of claim 84 wherein the step of synthesizing the RNA molecule is by chemical synthesis.

94. The method of claim 84 wherein the step of synthesizing the RNA molecule is by in vitro transcription or viral replication.

95. The method of claim 84 wherein the RNA molecule is a viral genome.

96. The method of claim 84 wherein the DNA-RNA hybrid is formed by repeated steps of (1) in vitro transcription from a double-stranded DNA template molecule and (2) reverse transcription of the RNA molecule product of (1)

97. The method of claim 96 wherein the repeated steps are repeated at least once.

98. The method of claim 96 wherein the double-stranded DNA molecule is a cDNA molecule generated from the reverse transcription and polymerase chain reaction of a mRNA molecule using a primer comprising nucleic acid sequence for the RNA polymerase promoter.

99. The method of claim 96 wherein the double-stranded DNA is generated by hybridization of chemically synthesized DNA sequences.

100. The method of claim 96 wherein the double-stranded DNA is a plasmid or viral vector.

101. The method of altering a characteristic of a eukaryotic cell, the method comprising introducing into the eukaryotic cell a nucleic acid molecule mixture comprising a first strand DNA molecule and a second strand RNA molecule, wherein the RNA molecule is complementary to the DNA.

102. The method of altering a characteristic of a eukaryotic cell, wherein either the RNA or the DNA molecule is complementary to a messenger RNA species in the cell.

103. The method of claims 89 or 92, wherein the synthesized DNA molecule contains none or at least one ribonucleotide or its analog.

104. The method of claims 93 or 94, wherein the synthesized RNA molecule contains none or at least one deoxyribonucleotide or its analog.

105. The method of claim 104, wherein the synthesized RNA molecule containing at least one deoxyribonucleotide analog is to increase RNAi phenomenon induction and to reduce interferon-related non-specific effects.
